(19) United States
(12) Patent Application Publication
(10) Pub. No.: US 2021/0338717 A1
(43) Pub. Date: Nov. 4, 2021

(54) NANOPARTICLE DELIVERY TO A TARGET TISSUE

(71) Applicant: THE GOVERNING COUNCIL OF THE UNIVERSITY OF TORONTO, Toronto (CA)

(72) Inventors: Ben OUYANG, Toronto (CA); Yi-Nan ZHANG, Toronto (CA); Wilson POON, Toronto (CA); Warren Che War CHAN, Toronto (CA); Zachary P. LIN, Richmond Hill (CA)

(73) Assignee: THE GOVERNING COUNCIL OF THE UNIVERSITY OF TORONTO, Toronto (CA)

(21) Appl. No.: 17/244,828

(22) Filed: Apr. 29, 2021

Related U.S. Application Data
(60) Provisional application No. 63/017,322, filed on Apr. 29, 2020.

(51) Int. Cl. A61K 33/242; A61K 47/69; A61K 9/14; A61P 35/00; A61K 47/02; A61K 33/00

(52) U.S. Cl. CPC A61K 33/242; A61K 47/6929; A61K 33/00; A61P 35/00; A61K 47/02; A61K 9/146

(57) ABSTRACT

A composition including a mixture of: (a) first nanoparticle carrier vehicles loaded with an active ingredient for treating or preventing a disorder (therapeutic nanoparticles), and (b) second nanoparticle carrier vehicles without the active ingredient (decoy nanoparticles). Also method of treating or preventing a disease, disorder or condition comprising administering to a human subject in need a dose of a composition having nanoparticles containing an active ingredient effective to treat or prevent the disease, disorder or condition. The dose is in terms of number of nanoparticles, and the nanoparticles is one of gold nanoparticles, liposomes, silica nanoparticles, micelles, hydrogels or polymeric nanoparticles. The number of nanoparticles in the dose is of at least one and one-half (1.5) quadrillion (10¹⁵) nanoparticles.

### CROSS-REFERENCE TO RELATED APPLICATION

This application claims priority under 35 U.S.C. § 119(e) to U.S. Provisional Application No. 63/017,322, filed Apr. 29, 2020, the content of which is hereby incorporated by reference in its entirety.

### FIELD OF THE INVENTION

The present invention relates in general to nanoparticle delivery to a target tissue.

### BACKGROUND OF THE INVENTION

Effective therapies for the treatment of solid tumours require efficient delivery of the injected medicine to the tumour. A single nanoparticle can pack tens of thousands of drug molecules and is one of the most studied delivery systems to treat cancer. However, only 0.7% (median) of administered nanoparticles are delivered to the solid tumour [1]. This low delivery efficiency has contributed to the poor clinical translation of nanoparticles for solid tumour treatments.

The liver is one of the major barriers inhibiting delivery of nanoparticles larger than 10 nm to solid tumours. The liver specializes in removing foreign materials from the blood and it can sequester 30-99% of the injected nanoparticle dose [2]. Liver cells such as Kupffer cells are responsible for this clearance [3,4] through various cellular mechanisms [5-7]. A major focus of nanoparticle research has been to avoid liver clearance by reducing these interactions. Such strategies have focused on chemically modifying nanoparticle size [8-10], shape [11,12], and surface chemistry [13-15], or biologically modifying Kupffer cells [16,17]. These efforts have reduced nanoparticle-liver interactions but have only improved tumour delivery to 2% [17].

### SUMMARY OF THE INVENTION

In one embodiment, the present invention relates to a composition comprising a mixture of: (a) first nanoparticle carrier vehicles loaded with an active ingredient for treating a disorder (therapeutic nanoparticles), and (b) second nanoparticle carrier vehicles without the active ingredient (decoy nanoparticles), the nanoparticle carrier vehicles being one of gold nanoparticles, liposomes, silica nanoparticles, micelles, nanogel or polymeric nanoparticles.

In one embodiment of the composition of the present invention, other than the presence of the active ingredient in the therapeutic nanoparticles, a chemical composition of the therapeutic nanoparticles is substantially similar or substantially identical to a chemical composition of the decoy nanoparticles.

In another embodiment of the composition of the present invention, the therapeutic nanoparticle is loaded with a single active ingredient for treating or preventing a disorder.

In another embodiment of the composition of the present invention, the decoy nanoparticle is devoid of any biologically active ingredient.

In another embodiment of the composition of the present invention, the composition comprises a higher ratio of decoy:therapeutic nanoparticles to stoichiometrically force the decoy nanoparticles to bind to liver cells responsible for nanoparticle clearance thereby allowing the therapeutic nanoparticles for continued circulation.

In another embodiment of the present invention, the composition comprises a combined dose for humans of therapeutic nanoparticles and decoy nanoparticles of at least one and one-half (1.5) quadrillion (10¹⁵) nanoparticles.

In another embodiment of the composition of the present invention both the therapeutic and decoy nanoparticles include a polyethylene glycol coating.

In another embodiment, the present invention is a combination medicament comprising: (a) a first composition comprising nanoparticle carrier vehicles loaded with an active ingredient for treating a disorder (therapeutic nanoparticles), and (b) a second composition comprising nanoparticle carrier vehicles without the active ingredient (decoy nanoparticles), wherein a chemical composition of the therapeutic nanoparticles in the first composition is substantially similar to a chemical composition of the decoy nanoparticles in the second composition, the nanoparticle carrier vehicles being one of silica nanoparticles, gold nanoparticles, liposomes, micelles, nanogel or polymeric nanoparticles.

In one embodiment of the combination medicament of the present invention, other than the presence of the active ingredient in the therapeutic nanoparticles, a chemical composition of the therapeutic nanoparticles is substantially similar to a chemical composition of the decoy nanoparticles.

In another embodiment of the combination medicament of the present invention, the therapeutic nanoparticle is loaded with a single active ingredient for treating or preventing a disorder.

In another embodiment of the combination medicament of the present invention, the decoy nanoparticle is devoid of any biologically active ingredient.

In another embodiment of the combination medicament of the present invention, the composition comprises a higher ratio of decoy:therapeutic nanoparticles to stoichiometrically force the decoy nanoparticles to bind to liver cells responsible for nanoparticle clearance thereby allowing the therapeutic nanoparticles for continued circulation.

In another embodiment of the present invention, the combination medicament comprises a combined dose for humans of therapeutic nanoparticles and decoy nanoparticles of at least one and one-half (1.5) quadrillion (10¹⁵) nanoparticles.

In another embodiment of the combination medicament of the present invention the therapeutic nanoparticles in the first composition and the decoy nanoparticles in the second composition include a polyethylene glycol coating.

In another embodiment, the present invention is a kit comprising: (a) a composition comprising first nanoparticle carrier vehicles loaded with an active ingredient for treating a disorder (therapeutic nanoparticles), (b) a composition comprising second nanoparticle carrier vehicles without the active ingredient (decoy nanoparticles), and (c) instruction for administering composition (a) and composition (b), the nanoparticle carrier vehicles being one of silica nanoparticles, gold nanoparticles, liposomes, micelles, nanogel or polymeric nanoparticles.

In one embodiment of the kit of the present invention, other than the presence of the active ingredient in the therapeutic nanoparticles, a chemical composition of the therapeutic nanoparticles is substantially similar to a chemical composition of the decoy nanoparticles.

In another embodiment of the kit of the present invention, the therapeutic nanoparticle is loaded with a single active ingredient for treating or preventing a disorder.

In another embodiment of the kit of the present invention, the decoy nanoparticle is devoid of any biologically active ingredient.

In another embodiment of the kit of the present invention, the instruction includes administering a higher ratio of decoy:therapeutic nanoparticles to stoichiometrically force the decoy nanoparticles to bind to liver cells responsible for nanoparticle clearance thereby allowing the therapeutic nanoparticles for continued circulation.

In another embodiment of the kit of the present invention, the instruction includes administering a combined dose for humans of therapeutic nanoparticles and decoy nanoparticles of at least one and one-half (1.5) quadrillion (10¹⁵) nanoparticles.

In another embodiment of the kit of the present invention, the instructions include instructions for co-administering composition (a) and composition (b) simultaneously.

In one embodiment of the kit according to any of the previous embodiments, composition (a) and composition (b) are provided in a single mixture.

In one embodiment of the kit according to any of the previous embodiments, the therapeutic nanoparticles and the decoy nanoparticles include a polyethylene glycol coating.

In one embodiment of the composition, the combination medicament, or the kit according to any one of the previous embodiments, the decoy nanoparticles are loaded with an agent that is different from the active ingredient of the therapeutic nanoparticles.

The present invention, in another embodiment, provides a use of the compositions, medicaments or kits of the present invention to treat or prevent a disease, disorder or condition. In one embodiment, the disease, disorder or condition is cancer or a non-cancer disease, disorder or condition.

In one embodiment, the present invention is a method of increasing dose efficacy or delivery efficiency for a therapeutic treatment of a disease, disorder or condition in a subject, the method comprising simultaneously co-administering to the subject a composition or combination medicament according to an embodiment of the present invention.

In another embodiment, the present invention is a method of treating a disease, disorder or condition, the method comprising simultaneously co-administering to a subject in need a composition or combination medicament according to the present invention.

In another embodiment, the present invention is a method of treating a disease, disorder or condition, the method comprising administering to a human subject in need a dose of a composition or combination medicament according to the present invention in terms of number of nanoparticles. In one aspect, the number of nanoparticles in the dose is of at least one and one-half (1.5) quadrillion (10¹⁵) nanoparticles. In another aspect of this embodiment, the dose is provided as a single dose.

In one embodiment of the present invention, according to any of the previous embodiments, the disease, disorder or condition is cancer.

### BRIEF DESCRIPTION OF THE DRAWINGS

The following figures illustrate various aspects and preferred and alternative embodiments of the invention.

FIGS. 1A-1E. Liver clearance of nanoparticles depends on dose. (FIG. 1A) Liver accumulation of PEGylated gold nanoparticles 24 hours post injection as a function of dose determined using ICP-MS. n=6. (FIG. 1B) Nanoparticle blood kinetics as a function of dose. n=3. (FIG. 1C) Half-life of nanoparticles as a function of dose. Numbers beside data points indicate dose in trillions of particles. n=3. (FIG. 1D) In vitro macrophage uptake over 24 hours as a function of dose. Uptake stopped increasing at approximately 100,000 nanoparticles per cell per 24 hours. n=3. (FIG. 1E) Quantification of nanoparticle signal in liver F4/80+ macrophages and autofluorescent+hepatocytes using histology. Kupffer cell uptake slowed first, then hepatocytes (inset) took up nanoparticles. n=60 cells from 3 mice.

FIGS. 2A-2I. High doses overwhelm liver uptake rates. (FIG. 2A) Live intravital imaging of Csf1r-EGFP mice livers. Mice were injected with 0.2 trillion Cy3-gold nanoparticles (red). Single EGFP+ Kupffer cells (blue) were imaged over 25 minutes to determine gold nanoparticle uptake. (FIG. 2B) Intravital imaging of Csf1r-EGFP mice livers. Mice were co-injected with 0.2 trillion Cy3-gold nanoparticles (red) and 12 trillion Cy5-gold nanoparticles (green). Top: imaging in the Cy3 channel. Bottom: imaging in the Cy5 channel. (FIGS. 2C-2D) Total uptake of Cy3-labelled nanoparticles over 25 minutes, from timelapses in FIG. 2A and FIG. 2B. Symbols represent individual Kupffer cells. n=9-12 Kupffer cells from 3 mice. (FIG. 2E) Quantification of uptake rates over 25 minutes using the slopes of FIGS. 2C-2D. Bars represent mean±s.e.m. n=9-12 Kupffer cells from 3 mice. Statistical significance was evaluated using a two-tailed unpaired t-test. **** p<0.0001. (FIG. 2F) Total uptake of Cy5-labelled nanoparticles over 60 minutes in the same Kupffer cells in FIG. 2B. (FIGS. 2G-2I) Imaging of liver CD209+ sinusoids (green) 15 minutes after injection. Darkfield+nanoparticle (red) distributed along walls for low dose (0.8 trillion) and distributed in the lumen for high dose (50 trillion). Scale bar: 40 μm. Lines with shading represent mean±s.e.m. n=60 blood vessel cross-sections from 3 mice. All data points and error bars represent mean±s.e.m.

FIGS. 3A-3H. Tumour delivery depends on dose. (FIG. 3A) Tumour delivery as a function of dose. An inflection occurred around 1 trillion nanoparticles (grey zone, calculated with limits of uptake by in vitro macrophages). Numbers indicate nanoparticle diameters, in nanometres. n=6 for 50 nm nanoparticles and n=3 for 15 nm and 100 nm nanoparticles. R2=0.74. (FIG. 3B) Liver accumulation as a function of dose behaved in an inverse pattern to the tumour, with inflection occurring at the same dose range. n=6 for 50 nm nanoparticles and n=3 for 15 nm and 100 nm nanoparticles. R2=0.86. High doses increased tumour delivery and reduced liver accumulation in 4 different mouse models, including (FIG. 3C) xenogeneic MDA-MB-231 mammary carcinoma in CD-1 nude mice (low dose (0.2 trillion) n=7, high dose (50 trillion) n=6), (FIG. 3D) transgenetic MMTV-PyMT mammary carcinoma in FVB/n mice (low dose tumours n=39, livers n=4; high dose tumours n=40, livers n=3; note that sample numbers are inconsistent due to differences in tumour growth and a liver sample processing accident), (FIG. 3E) xenogeneic U87 glioma in CD-1 nude mice (n=3), and (FIG. 3F) orthotopic 4T1 in CD-1 nude mice (n=3). High doses increased tumour delivery and reduced liver accumulation in a different nanoparticle (FIG. 3G) fluorescently-labelled silica nanoparticles (n=3). In (k), a medium dose (3.1 trillion) was chosen because fluorophore conjugation efficiency on silica particles was too low to be measured using the low dose. High doses increased tumour delivery and reduced liver accumulation in a nanoparticle (FIG. 3H) radiolabelled liposomes (n=5-6). All data points and error bars represent mean±s.e.m. Statistical significance was evaluated using a two-tailed unpaired t-test. * p<0.05, *** p<0.001, **** p<0.0001.

FIGS. 4A-4H. Tumour penetration and cell delivery depends on dose. (FIG. 4A) Representative 3D volumes of tumours of mice injected with 2 trillion or 12 trillion nanoparticles. Blood vessels in red, and nanoparticles in green. (FIG. 4B) Nanoparticle diffusion distance from walls of blood vessels for 2 and 12 trillion nanoparticles. Intensity was normalized to the intensity at the vessel wall. n=4 samples from 2 mice in 2 trillion dose. N=6 samples from 3 mice in 12 trillion dose. Lines and shadings represent mean±s.e.m. (FIG. 4C) Representative histology sections of tumours of mice injected with a low dose (0.2 trillion) or a high dose (50 trillion) of nanoparticles after 24 hours of circulation. Nuclei are represented in blue (fluorescence) and nanoparticles in green (darkfield imaging). (FIG. 4D) Representative transmission electron microscopy images of tumours of mice injected with a high dose of nanoparticles after 24 hours of circulation. Most nanoparticles were intracellular. (FIG. 4E) Flow cytometry gating strategy for investigating cells that took up nanoparticles. (FIG. 4F) Representative flow cytometry plots of live cells in the tumour that took up nanoparticles as a function of dose. (FIG. 4G) Quantification of flow cytometry plots in FIG. 4D for the proportion of live cells in the tumour that took up nanoparticles as a function of dose. n=3. (FIG. 4H) The amount of nanoparticles taken up per cell, expressed in terms of mean fluorescent intensity. n=3. Data points represent mean±s.e.m.

FIGS. 5A-5E. Gold nanoparticle characterization. (FIG. 5A) Schematics of gold nanoparticles ligand-stabilized using 5k mPEG and fluorescently labelled with 10k amine-terminated poly(ethylene glycol) conjugated to Cy5 in 3 sizes: (i) 15 nm, (ii) 50 nm, (iii) 100 nm. (FIG. 5B) transmission electron microscopy images of (i) 15 nm, (ii) 50 nm, (iii) 100 nm gold nanoparticles. Scale bars as indicated in images. (FIGS. 5C&5E) Dynamic light scattering measurements of hydrodynamic diameters of gold nanoparticles. (FIGS. 5D*5E) UV-visible spectroscopy of gold nanoparticles. The absorption shoulder at 647 nm is the absorbance band of Cy5, which is visible over the gold absorbance mostly in 15 nm nanoparticles and some in 50 nm nanoparticles.

FIGS. 6A-6H. Dose determined delivery efficiency in publications from 2005-2015. Studies from the meta-analysis by Wilhelm et al.1 were evaluated to determine the effect of dose in mice. (FIG. 6A) Delivery efficiency as a function of dose across n=67 data points from 40 publications between 2005 and 2015. There was a positive correlation between dose and delivery efficiency (R2=0.39, p<0.0001). Gray bar represents in vitro uptake threshold described in FIG. 1d. (FIG. 6B) The data in FIG. 6A was divided into two halves by the median dose of the publications: 1.2 trillion nanoparticles. The data from (FIGS. 6A-6B) were stratified according to (FIGS. 6C-6D) organic vs inorganic classes of nanoparticles, (FIGS. 6E-6F) passive vs active targeting nanoparticles and (FIGS. 6G-6H) small vs large nanoparticles. Bars represent mean±s.e.m. Statistical significance was evaluated using a two-tailed unpaired t-test. * p<0.05, ** p<0.01, *** p<0.001.

FIGS. 7A-7C. Liver macrophage uptake quantification by histology. Representative images used for quantification in FIG. 1 E. Macrophages were manually traced around F4/80+ (red) regions in ImageJ, and this was used as a mask to quantify the nanoparticle (white) accumulation. Nanoparticles of 20 randomly-selected macrophages were quantified per slide. Average nanoparticle signal per cell was divided by the dose of nanoparticles injected to obtain graph in FIG. 1E. FIG. 7A illustrates liver macrophage uptake of nanoparticles and an enlarged view at a dose of 50 trillion nanoparticles; FIG. 7B at a dose of 3.1 trillion nanoparticles; and FIG. 7C at a dose of 0.2 trillion nanoparticles.

FIGS. 8A-8B. Hepatocyte nanoparticle uptake beyond dose threshold. Representative images used in quantification in FIG. 1e (inset). (FIG. 8A) Hepatocytes (green) took up nanoparticles (white) at a bolus dose of 50 trillion (yellow arrows). (FIG. 8B) Nanoparticles in hepatocytes were undetectable at doses lower than 1 trillion.

FIGS. 9A-9B. Flow cytometry live/dead analysis of liver cells at different doses. (FIG. 9A) Quantification of live cells in the liver of mice administered with a low dose (0.2 trillion) or high dose (50 trillion) PEGylated gold nanoparticles, 24 hours after injection. Survival was normalized to the proportion of live cells in the low dose condition. High dose live cell proportion was not different than low dose live cell proportion (two-tailed unpaired t-test, p=0.6784). (FIG. 9B) Representative gating strategy used to identify proportion of live cells.

FIGS. 10A-10E. Cytotoxicity of gold nanoparticles at low and high doses. There was no observable effect of dose on the cytotoxicity of macrophages and hepatocytes in the range of doses studied. (FIG. 10A) gating strategy used to identify macrophages and hepatocytes. (FIG. 10B) a live/dead stain was used to identify live macrophages. (FIG. 10C) a cryosection of liver dosed with low or high numbers of nanoparticles. Blue is DAPI, red is F4/80 and nanoparticles, and green is autofluorescence (hepatocytes). (FIG. 10D) a live/dead stain was used to identify live hepatocytes. (FIG. 10E) a paraffin-fixed section of liver dosed with low or high numbers of nanoparticles. H&E stained.

FIGS. 11A-11D. Histology of liver at high and low doses of gold nanoparticles. (FIGS. 11A-11B) Representative image of a liver of a mouse administered with a low dose (0.2 trillion) of gold nanoparticles, stained with (FIG. 11A) H&E and (FIG. 11B) TUNEL. (FIGS. 11C-11D) Representative image of a liver of a mouse administered with a high dose (50 trillion) of gold nanoparticles, stained with (FIG. 11C) H&E and (FIG. 11D) TUNEL. The dark purple are gold nanoparticles that have accumulated in the sinusoidal cells. Note that no nuclei have stained positive for TUNEL.

FIG. 12. Blood clearance of daily repeated low dose injections. Gold nanoparticles were injected daily (arrows). Blood was collected at the following timepoints: just before injection, 10 minutes, 1 hour, 4 hours. On day 4, there was a sudden acceleration of blood clearance, in agreement with the accelerated blood clearance (ABC) phenomenon. All data points and error bars represent the mean±s.e.m. (n=3).

FIGS. 13A-13C. Non-normalized uptake intensities of liver cells. Additional graphs for FIG. 1E without dose normalization. (FIG. 13A) Liver cell uptake (blue, red) compared against a linearly-increasing trend, by extrapolating from the linear intensity increase in liver macrophages in doses up to 0.80 trillion nanoparticles. (FIG. 13B) Zoom in of FIG. 13A. Kupffer cell uptake was proportionally less with increasing dose, but not completely saturated. (FIG. 13C) Zoom in of FIG. 13B. Hepatocyte uptake of nanoparticles increased with increasing dose. All data points and error bars represent mean±s.e.m. n=30 cells from 3 mice.

FIGS. 14A-14B. Nanoparticle uptake capacity of macrophages. (FIG. 14A) Representative TEM imaging of a Kupffer cell in a liver sinusoid 30 minutes after nanoparticle injection. Note that the nanoparticles occupied a minor proportion of the Kupffer cell volume. KC: Kupffer Cell, RBC: Red Blood Cell. (FIG. 14B) TEM imaging of a RAW264.7 macrophage incubated with nanoparticles for 24 hours, demonstrating the large nanoparticle uptake capacity of macrophages.

FIGS. 15A-15B. Nanoparticles bind to membranes. (FIG. 15A) Representative TEM images of Kupffer cells in liver sinusoids, 30 minutes after nanoparticle injection. Images on the right are higher magnifications. Nanoparticles are seen to line the perimeters of Kupffer cell endosomes, suggesting they were internalized after binding to the Kupffer cell membrane. Note the density of packing onto the endosomal membranes, with minimal available binding sites. (FIG. 15B) Uptake into RAW264.7 macrophages in vitro shows similar pattern of endosome membrane binding.

FIGS. 16A-16B. Gold nanoparticle co-localization with 70 kDa dextran. (FIG. 16A) Representative images of live single-cell in vivo intravital images, 1 hour after co-injection of 0.2 trillion gold nanoparticles with 1 mg/mL dextran-70 kDa. Kupffer cells (blue) took up gold C3-labelled gold nanoparticles (red) and Cy5-labelled 70 kDa dextran (green) into different subcellular locations. (FIG. 16B) Quantification of co-localization of gold and dextran by Pearson's r and Mander's M coefficients. Low correlation suggested gold nanoparticles were taken up by a different pathway than macropinocytosis (dextran).

FIGS. 17A-17E. Compartment modelling predicts kinetic delivery to tumours. (FIG. 17A) Compartment model. Nanoparticles injected into the blood transfer into peripheral organs (liver, tumour, and others) with individual rate constants k. Nanoparticle transfer back into blood was assumed to be negligible. kL,KC, transport rate constant from blood to liver Kupffer cells, kL,OC, transport rate constant from blood to liver other cells, kT, transport rate constant from blood to tumour, kO, transport rate constant from blood to other organs. (FIGS. 17B-17C) Simulated accumulation rates in the liver (black) and tumour (red) when given a low dose of 0.2 trillion nanoparticles (FIG. 17B) and high dose of 50 trillion nanoparticles (FIG. 17C). (FIGS. 17D-17E) Total accumulation in the liver (gray, black) and tumour (pink, red) when given a low dose (FIG. 17D) and high dose (FIG. 17E) of nanoparticles. Experimental data at 2 hours, 8 hours, and 24 hours post injection overlaid onto simulated data. n=3 for each experimental point. All data points and error bars represent mean±s.e.m.

FIGS. 18A-18E. Single versus repeated dosing. (FIG. 18A) A single dose injection of 5 trillion nanoparticles (above the 1 trillion threshold) into 4T1-tumour bearing BALB/c mice was compared with 8 daily doses of 0.625 trillion nanoparticles (below the 1 trillion threshold). Total dose in both groups was 5 trillion. Mice were inoculated with tumour cells, and 7 days later, were injected daily with 0.625 trillion nanoparticles. On day 14, the single dose group of mice were injected with 5 trillion nanoparticles. On day 15, mice were sacrificed for biodistribution analysis. (FIG. 18B) Half-life of bolus doses was longer than that of repeated doses. (FIGS. 18C-18D) Total tumour delivery of bolus doses was more than that of repeated doses. Gold nanoparticle accumulation in tumours was visible by eye in ex vivo tumours (purple). (FIG. 18E) Total liver accumulation of bolus doses was less than that of repeated doses. Bars represent mean±s.e.m. n=4 for the repeated dose, n=3 for the single dose. Statistical significance was evaluated using a two-tailed unpaired t-test. * p<0.05, ** p<0.01, *** p<0.001, ****p<0.0001.

FIGS. 19A-19E. Silica nanoparticle characterization. Silica nanoparticles were studied as another inorganic nanoparticle to explore the universality of nonlinear dose responses. (FIG. 19A) schematics and synthesis summary of silica nanoparticles. (FIG. 19B) Transmission electron microscopy of silica particles 50 nm in diameter. (FIG. 19C) Dynamic light scattering of bare particles and particles conjugated with surface ligands and fluorophores. (FIG. 19D) UV-visible spectroscopy of bare silica nanoparticles and silica nanoparticles conjugated with Cy5. (FIG. 19E) Fluorescence emission spectra of bare and Cy5-conjugated silica nanoparticles with excitation at 647 nm.

FIGS. 20A-20K. Biodistribution of 50 nm gold nanoparticles in internal organs. Supplemental organs to FIGS. 3A-3B. BALB/c mice with 2-week old 4T1 tumours were injected intravenously with varying doses of 50 nm gold nanoparticles, then sacrificed 24 hours later. Organs were excised and quantified for gold nanoparticle accumulation using ICP-MS. Y-axis was set to a maximum of 60% injected dose/gram, as in FIG. 1A. Notably, splenic accumulation was not correlated with dose. n=3. All data points represent mean±s.e.m.

FIGS. 21A-21D. Dose alternative normalization. Supplementary figure to FIGS. 3A-3B. (FIGS. 21A-21B) Dose was renormalized by surface area in cm² for tumours (FIG. 21A) and livers (FIG. 21B). Correlation between surface area dose and % injected dose/g was lower than correlation between number dose and % injected dose/g. (FIGS. 21C-21D) Dose was renormalized by mass in mg of gold for tumours (FIG. 21C) and livers (FIG. 21D). Correlation between mass dose and % injected dose/g was lower than correlation between number dose and % injected dose/g. This further confirmed that dose by number of nanoparticles is the most appropriate standard unit for dose.

FIG. 22. Dose-dependency in Kupffer cell depleted mice. Mice were pre-treated with clodronate liposomes 2 days before gold nanoparticle injection (50 nm) to deplete Kupffer cells. Tumour delivery was measured 24 hours after gold nanoparticle injection. Low dose, 0.2 trillion; high dose, 50 trillion. Bars represent mean±s.e.m. Statistical significance was evaluated using a two-tailed unpaired t-test.

FIGS. 23A-23E. Specificity of dose enhancement. Delivery enhancers (46 trillion) were co-injected with a low dose (0.2 trillion) of gold nanoparticles to investigate if dose enhancement required nanoparticle specificity. (FIG. 23A) experimental schematics. (FIG. 23B) Two groups were investigated: low dose of gold nanoparticles alone versus low dose of gold nanoparticles with delivery enhancers. (FIGS. 23C-23E) The tumour and liver were not statistically different in their accumulation of gold nanoparticles. The amount of nanoparticles remaining in endpoint blood at 24 hours was statistically different (** p<0.01). Data points represent mean±s.e.m (n=3). Statistical significance was evaluated using a two-tailed unpaired t-test.

FIGS. 24A-24B. Gold nanoparticle and liposome uptake by Kupffer cells. Intravital liver imaging depicting single-cell images of live Kupffer cells in vivo. (FIG. 24A) Timelapse uptake of liposomes (red) and gold nanoparticles (green) in Kupffer cells (blue) over 1 hour. (FIG. 24B) Higher magnification of live Kupffer cells in vivo at 1 hour after injection demonstrating that some Kupffer cells predominantly take up liposomes (arrowhead) and some Kupffer cells take up only gold nanoparticles (arrows).

FIGS. 25A-25E. Composition of adsorbed proteins on gold nanoparticles and liposomes. (FIG. 25A) Experimental schematic. The liposomes and gold nanoparticles were first incubated with the pooled mouse serum for one hour at 37° C. to allow serum proteins to adsorb. The protein-liposome complexes were separated from unbound proteins by size exclusion chromatography and concentrated with ultra-centrifugation. The protein-gold nanoparticle complexes were separated through centrifugation washing three times. Next, the adsorbed proteins were isolated from the nanoparticle surface and trypsinized into peptides. They were then identified and quantified on LC-MS/MS. (FIGS. 25B-25C) The top 20 adsorbed proteins identified on gold nanoparticles and liposomes. (n=3, Pearson correlation between replicates AuNPs: 0.93, Liposomes: 0.77). (FIG. 25D) Venn diagram of shared and unique proteins on gold nanoparticles and liposomes. (FIG. 25E) A volcano plot of proteins adsorbed on gold nanoparticles and liposomes. Proteins highlighted in green were statistically significantly more abundant on gold nanoparticles. LAC1 is the Ig lambda-1 chain C region. HVM32 is the Ig heavy chain V-III region J606. IGHG3 is the Ig gamma-3 chain C region. CO3 is the Complement C3. GCAM is Ig gamma-2A chain C region, membrane-bound form. (Red line: p<0.05).

### DESCRIPTION OF THE INVENTION

Definitions

The practice of the present invention will employ, unless otherwise indicated, conventional techniques of tissue culture, immunology, molecular biology, microbiology, and cell biology which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in

Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Patent No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

All numerical designations, e.g., pH, temperature, time, concentration and molecular weight, including ranges, are approximations which are varied (+) or (−) by increments of 1.0 or 0.1, as appropriate, or alternatively by a variation of +/−15%, or alternatively 10%, or alternatively 5% or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a polypeptide” includes a plurality of polypeptides, including mixtures thereof.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention. Embodiments defined by each of these transition terms are within the scope of this invention.

As used herein, “co-administration” means simultaneous administration, and it includes the administration of a single composition comprising the decoy nanoparticles and the nanoparticles carrying the active agent, or the co-administration of two compositions, a first composition comprising the decoy nanoparticles, and a second composition comprising the nanoparticles carrying the active agent being administered simultaneously. Of course, the first composition may or may not include nanoparticles carrying the active agent, and the second composition may or may not include decoy nanoparticles. Simultaneous administration means that the second composition is administered in less than 1.48 hours after the administration of the first composition. More preferable the second composition is administered in less than 15 minutes after the administration of the first composition. More preferable the second composition is administered in less than 10 minutes after administration of the first composition. In another embodiment the second composition is administered zero (0) minutes after the administration of the first composition.

The term “effective amount” as used herein refers to an amount sufficient to induce a detectable therapeutic response in a subject.

The term “substantially” includes exactly the term it modifies and slight variations therefrom.

As used herein, the terms “treating,” “treatment” and the like are used herein to mean obtaining a desired pharmacologic and/or physiologic effect. The effect may be prophylactic in terms of completely or partially preventing an infection, disorder, disease, condition or sign or symptom thereof and/or may be therapeutic in terms of a partial or complete cure for an infection, a disease, a disorder and/or adverse effect attributable to the infection, disease or disorder.

To “prevent” intends to prevent an infection or disorder or effect in vitro or in vivo in a system or subject that is predisposed to the disorder or effect.

A nanogel is a nanoparticle composed of a hydrogel—a crosslinked hydrophilic polymer network. Nanogels are most often composed of synthetic polymers or biopolymers which are chemically or physically crosslinked. Nanogels are usually in the tens to hundreds of nanometers in diameter. The pores in nanogels can be filled with small molecules or macromolecules, and their properties, such as swelling, degradation, and chemical functionality, can be controlled.

Polymeric nanoparticles (PNP) are defined as sub-micron (1 to 1000 nm) colloidal particles with or without active pharmaceutical ingredients encapsulated within via adsorption to macromolecular substances (polymer) via Van der Waals and/or electrostatic interactions.

Overview

Disclosed herein is a method for Artificially Increasing Dose for Efficacy (AIDE) for a therapy in which an active agent or drug, encapsulated in an inorganic nanoparticle carrier vehicle or a polymeric nanoparticle system, such as a gold nanoparticle or a silica nanoparticle, is supplemented by administration of a nanoparticle carrier vehicle without active agent or drug (decoy or blank). In a tumour model for mammary carcinoma, AIDE produced greater therapeutic effect than the sum of the effects from administration of each component alone, as shown by smaller tumour sizes and prolonged survival time.

Compositions

The present invention provides for a composition comprising: (a) first nanoparticle carrier vehicles loaded or comprising an active ingredient(s) for treating a disorder, such as an anticancer agent(s) (therapeutic nanoparticles) and (b) second nanoparticle carrier vehicles without the active ingredient(s) (decoy nanoparticles). The nanoparticle is a gold nanoparticle, liposome, a silica nanoparticle, a micelle, a nanogel or a polymeric nanoparticle.

In one embodiment, the chemical composition of the first nanoparticle carrier vehicles in (a) is substantially similar or identical to the chemical composition of the second nanoparticle carrier vehicles.

In another embodiment both the first and second nanoparticle carrier vehicles include polyethylene glycol coatings (PEGylation).

In another embodiment, the present invention provides for a combination medicament, the combination medicament comprising two compositions: a first composition comprising therapeutic nanoparticles and a second composition comprising decoy nanoparticles. In one aspect the decoy and therapeutic nanoparticles are PEGylated. In another aspect, the chemical composition of the decoy and the therapeutic nanoparticles are substantially similar or substantially identical.

In another embodiment, the present invention provides for a kit comprising: (a) a composition comprising an effective amount of first nanoparticle carrier vehicles comprising or loaded with an active ingredient(s) for treating a disorder, such as an anticancer agent(s), and (b) a composition comprising an effective amount of second nanoparticle carrier vehicles without the active ingredient(s), and (c) instruction for administering composition (a) and composition (b).

In one embodiment the instructions of the kit include instructions for simultaneously co-administering compositions (a) and (b).

In another embodiment, the chemical composition of first nanoparticle carrier vehicles in (a) is substantially similar or substantially identical to the chemical composition of the second nanoparticle carrier vehicles in (b).

In another embodiment, the chemical composition of the first nanoparticle carrier vehicles in (a) is different to the chemical composition of the second nanoparticle carrier vehicles in (b).

In one embodiment the first and second lipid based carrier vehicles include PEGylation.

The nanoparticle carrier vehicles of all the embodiments of the present invention can be, for example, lipid based carrier vehicles or lipid based drug delivery systems such as liposomes, or PLGA (poly(lactide-co-glycolic acid) and gold nanoparticles.

In another embodiment, the present invention provides for a kit comprising: (a) a composition comprising an effective amount of first PEGylated nanoparticle carrier vehicles comprising or loaded with an active ingredient(s) for treating a disorder, such as an anticancer agent(s), and (b) a composition comprising an effective amount of second PEGylated nanoparticle carrier vehicles without the active ingredient(s), and (c) instruction for administering composition (a) and composition (b). In one embodiment, the chemical composition of the first nanoparticle carrier vehicles in (a) is substantially similar or substantially identical to the chemical composition of the second nanoparticle carrier vehicles in (b). In another embodiment, the chemical composition of the first nanoparticle carrier vehicles in (a) is different to the chemical composition of the second nanoparticle carrier vehicles in (b). In one embodiment the kit further comprises instructions to co-administering (a) and (b) simultaneously.

In this disclosure, a reference that the chemical composition of the therapeutic nanoparticle is substantially similar or identical to the chemical composition of the decoy nanoparticle does not include any load being carried by the therapeutic nanoparticles and the decoy nanoparticles. That is, what is being compared is the chemical structure/composition of the nanoparticle vehicles themselves (i.e. the empty nanoparticles). That is, other than any load being carried by the therapeutic nanoparticles and the decoy nanoparticles, such as the active ingredient, the chemical composition or chemical structure of the therapeutic nanoparticles and the decoy nanoparticles themselves are substantially similar or even identical.

In one embodiment, the chemical composition or chemical structure of the decoy nanoparticle excludes ingredients that are biologically active.

The invention is further directed to pharmaceutical compositions comprising the therapeutic nanoparticle carrier vehicles, the decoy nanoparticle carrier vehicles and a pharmaceutically acceptable carrier. The pharmaceutical composition can further comprise an adjuvant. The adjuvant can enhance the biological activity of the one or more pharmaceutical agents in the pharmaceutical composition.

The pharmaceutical composition can be formulated for injection (e.g., intramuscular, subcutaneous, intravenous, or intraperitoneal injection), oral administration, topical administration, transdermal administration, intranasal administration, or inhalation.

Pharmaceutically acceptable carriers are well known to those skilled in the art and include, for example, sterile saline, lactose, sucrose, calcium phosphate, gelatin, dextrin, agar, pectin, peanut oil, olive oil, sesame oil, and deionised water.

The pharmaceutical composition can comprise one or more stabilizers. For example, the stabilizer can comprise a carbohydrate (e.g., sorbitol, mannitol, starch, sucrose, dextrin, glucose, or a combination thereof), a protein such as albumin or casein, and/or a buffer (e.g., an alkaline phosphate).

Compositions for injection may include one or more pharmaceutically acceptable vehicles or diluents. Compositions for injection can comprise buffered solutions that have a suitable pH and are iso-osmotic with physiological fluids. Any pharmaceutically suitable diluent may be used in the composition for injections (e.g., distilled water, a salt solution, and/or a buffer solution). Compositions for injection may be prepared by conventional volume-weight procedures. A certain amount of the mixture may be diluted to the necessary volume with a diluent or solvent. The solution may then filtered through sterilized filters and then bottled or ampouled.

The therapeutic nanoparticles of the present invention are loaded with at least one active ingredient that is useful in treating or preventing a disorder (including infections, diseases and conditions). For example, the therapeutic nanoparticles of the present invention may be loaded with anti-cancer agents such as paclitaxel, doxorubicin, including doxorubicin hydrochloride, doxorubicin sulfate, doxorubicin citrate, and/or any other suitable anti-cancer agent.

The decoy nanoparticles of the present invention may, in one embodiment, be empty nanoparticles (i.e. not carrying active agents inside, but may still be filled with a buffer or liquid or a solid salt solution). In another embodiment, the decoy nanoparticles of the present invention may be filled or loaded with an active ingredient that is different from the active ingredient in the therapeutic nanoparticle, or may be filled with adjuvants, flavoring agents, dyes, tracing labels, and so forth. For example, if the therapeutic nanoparticle is filled with an anti-cancer agent, the decoy nanoparticles may be filled with another anti-cancer agent, or with an immunosuppressant, and so forth.

Therapeutic Methods and Uses

The compositions of the present invention can be used in a number of therapeutic methods.

In one embodiment, the present invention provides for a method of treating a disorder in a subject in need comprising simultaneously co-administering to the subject (a) an effective amount of first nanoparticle carrier vehicles, such as liposomes, PLGA and gold nanoparticles, comprising or loaded with an active ingredient(s) for treating a disorder (including disease, infection and condition), such as an anticancer agent(s) and (b) an effective amount of second nanoparticle carrier vehicles without the active ingredient (i.e. decoy carrier vehicle). In one embodiment, the chemical composition of the first nanoparticle carrier vehicles is substantially similar or substantially identical to the chemical composition of the second nanoparticle carrier vehicles systems. In another embodiment, the chemical composition of the first nanoparticle carrier vehicles is different to the chemical composition of the second nanoparticle carrier vehicles. In one embodiment the first and second nanoparticle carrier vehicles include PEGylation. The nanoparticle carrier vehicle is one of gold nanoparticles, liposomes, a silica nanoparticles, micelles, nanogels or polymeric nanoparticles.

In another embodiment, the present invention provides for a method of treating a disorder comprising administering to a subject in need (a) an effective amount of therapeutic nanoparticle carrier vehicles, and (b) an effective amount of decoy nanoparticle carrier vehicles, wherein a chemical composition of the therapeutic nanoparticle carrier vehicles and the decoy nanoparticle carrier vehicles is substantially similar or identical.

In another embodiment, the present invention provides for a method of treating a disorder comprising administering to a subject in need (a) an effective amount of therapeutic PEGylated nanoparticle carrier vehicles, and (b) an effective amount of decoy PEGylated nanoparticle carrier vehicles.

In one embodiment of the methods of the present invention, the therapeutic nanoparticles are administered within about 15 minutes after administering the decoy nanoparticles. In another embodiment of the methods of the present invention, the therapeutic nanoparticles are administered together.

The (a) loaded and (b) decoy nanoparticle carrier vehicles of the present invention can be administered simultaneously as part of one composition, or administered independently in two independent compositions in a fashion such that the components of (a) and (b) act at the same time. Components administered independently can, for example, be administered separately (in time) or concurrently in time, at the same site of administration, at different sites of administration or via different routes of administration. Separately in time means, in one embodiment, no more than 1.48 hour apart. In another embodiment, separately in time means no more than about 15 minutes apart. In another embodiment separately in time means no more than 10 minutes. In the case of separate administration, the decoy nanoparticles can be administered before, during or after administration of the nanoparticles containing the active ingredient(s) at different sites or via different routes of administration. A worker skilled in the art can determine the elapsed time between the administration of the components of the invention when used in combination will be dependent upon, for example, the age, health, and weight of the recipient, nature of the combination treatment, side effects associated with the administration of other component(s) of the combination, frequency of administration(s), and the nature of the effect desired. Components of the combinations of the invention can also be administered independently with respect to location and, where applicable, route of administration in order to maximize the therapeutic benefit of each component. The composition(s) of the present invention can be administered via a single dose or via continuous perfusion. Non-limiting examples of route of administration include oral administration, nasal administration, injection (for example, subcutaneous, parenteral, intraperitoneal and intravenous injections) and topical application.

If 95% of the decoys are needed in the blood for optimal liver cell inhibiting activity and that decoy circulation half-life is 20 hours, then 1.48 hours will be the maximum time allowed to pass before there is less than 95% of decoys in the blood. That is the simultaneous co-administration would be 1.48 hours or less. If 99% of the decoys are needed in the blood, then the simultaneous window will be shortened to 16.8 minutes or less.

The methods and therapeutic uses of the present invention can further comprise administering another therapy (combination therapy). For example where the disease, disorder or condition comprises cancer, the methods of the present invention can further comprise administering a conventional cancer therapy to the subject. For example, the conventional cancer therapy can be selected from a cancer vaccine, chemotherapy, immunotherapy, radiation therapy or combinations thereof.

Advantages

(i) Use of PEGylated Nanoparticles

The use of PEGylated nanoparticles as decoys is inventive and non-obvious to use for the following reasons. PEGylated particles have low protein binding, and thus low affinity for the livers clearance cells. This allows the PEGylated nanoparticles to avoid liver clearance and circulate for longer. Therefore, PEGylated nanoparticles as decoys would not be an option to think about if someone were to choose a decoy particle. This is experimentally shown in Gabizon et al. (8) where a preinjection of PEGylated liposomes did not reduce clearance of a second injection of a tracer liposome. In the present disclosure it is shown, for the first time, that indeed, PEGylated liposomes can reduce clearance of another liposome of the same composition, and that these PEGylated liposomes did not reduce clearance of a PEGylated gold nanoparticle (of the same size). This is likely because the proteins that bind onto the surface of these particles are different for different particle types. Different bound proteins would lead particles to different cell receptors or cell types than the therapeutic particles, so they would have minimal effect. Since the decoys are the same as the therapeutic nanoparticle, they likely bind to the same receptors on the same cells, leading to a binding inhibition.

(ii) Simultaneous Co-Administration

The present invention provides for the simultaneous co-administration of decoy and therapeutic nanoparticle vehicles, as opposed to the sequential injections of the prior art.

Sequential injections must be optimized especially with a non-PEGylated nanoparticle. This is because the non-PEGylated nanoparticles have short half-lives (minutes to a few hours), and therefore need to be carefully studied and optimized to understand when they are blocking the liver.

To optimize the sequential injections, there must be an imaging aspect involving a method for visualizing the decoy nanoparticle, such as MRI, or sampling with biopsy. An imaging modality such as MRI requires an additional nanoparticle formulation for visualization and time on an MRI machine. These are prohibitive in terms of regulatory control, cost, and time. Biopsy requires invasive procedures to remove liver and/or tumour tissue from the patient. Although Liu et al (9) claimed it's easy to optimize (9), it is not easy.

Even if optimized, having two injections means the patient and clinical care teams must focus on the patient for two injections. Since nanoparticles are rarely delivered in bolus to humans and more as a continuous infusion, this would significantly lengthen the time of the procedure (e.g. one hour becomes two hours). Sequential injection is not practical.

The present invention also provides for a method of treating a disease, disorder or condition, the method comprising administering to a human subject in need a dose of a composition or combination medicament according to the present invention in terms of number of nanoparticles. In one aspect, for a human subject, the number of nanoparticles in the dose is of at least one and one-half (1.5) quadrillion (10¹⁵) nanoparticles.

The following example is intended to illustrate, but not limit the invention.

### EXAMPLES

### Example 1

### Nanomaterial Synthesis.

### Gold Nanoparticles

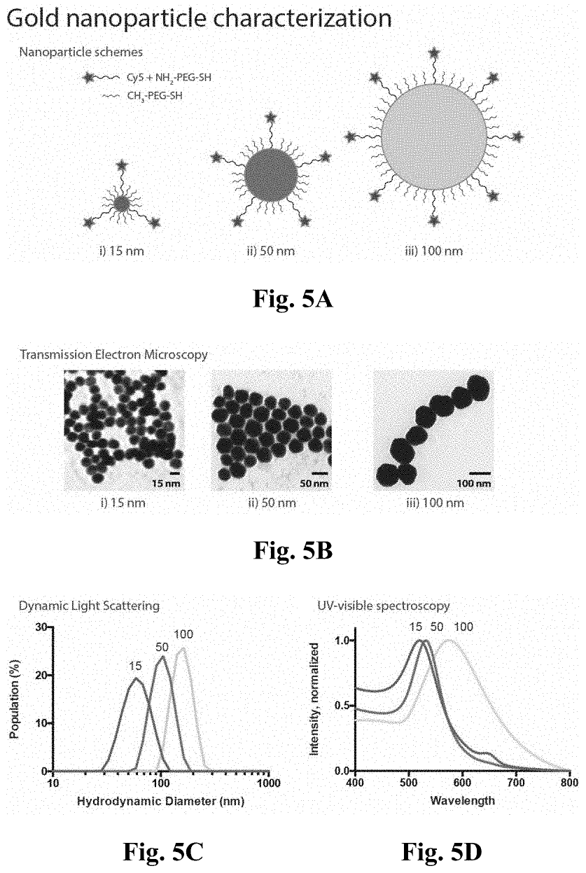

All glassware was cleaned using aqua regia (3:1 hydrochloric acid:nitric acid), dish soap, and deionized water before using. 15 nanometer particles were synthesized by adding 1 mL of 3% (w/v) sodium citrate tribasic dehydrate (Sigma S4641) to 98 mL of boiling deionized water under vigorous stirring, followed immediately by 0.1 mL of 10% (w/v) chloroauric acid tetrahydrate (Sigma 254169). This mixture was boiled and stirred for 7 minutes, and then immediately cooled in an ice water bath. 50 and 100 nm particles were synthesized by seed-mediated growths¹. 50 nm particles were synthesized by chilling reagents in an ice bath: 750 mL deionized water, 9.475 mL of 10% (w/v) (250 mM) chloroauric acid tetrahydrate, 9.629 mL of 4.41% (w/v) (150 mM) sodium citrate tribasic dehydrate, 250 mL of 4 nM 15 nm particles, 9.629 mL of 2.75% (w/v) (250 mM) hydroquinone (Sigma H17902). 100 nm particles were made by reducing the 250 mL of 15 nm particles to 23 mL of 15 nm and topping up with deionized water. The ice-cold reagents were added in the order listed above to a 2 L Erlenmeyer flask under vigorous stirring (note: hydroquinone was added as quickly as possible). The reaction proceeded overnight at 4° C. Synthesized nanoparticles were washed 2 times in a wash buffer containing 0.02% (w/v) sodium citrate tribasic+0.1% (w/v) Tween20. Washing proceeded as follows: 13 mL in a 15 mL centrifuge tube, centrifuged for 2 hours at 1500×g (50 nm) or 500×g (100 nm). Supernatant was discarded and particles were resuspended in 40 mL of wash buffer while under sonication in a water bath (Misonix 2510R-MT). This 40 mL was divided into forty 1.5 mL microcentrifuge tubes and centrifuged at the same speeds for 45 minutes. The supernatant was discarded and the particles resuspended in a total of 6 mL of wash buffer under sonication. All particles were characterized using UV-Vis spectroscopy, dynamic light scattering, and transmission electron microscopy (FIG. 5). Particles were PEGylated by mixing methoxy-terminated 5 kDa polyethylene glycol thiol (Laysan Bio Inc) and amine-terminated 10 kDa polyethylene glycol thiol (Rapp Polymere GmbH) in a 4:1 ratio. This mixture was added to the nanoparticles at a ratio of 5 PEG per nm², and incubated at 60° C. for 30 minutes. These PEGylated particles were washed 3 times in Eppendorf tubes with wash buffer (50 nm: 2500×g for 45 minutes). At the last step, the supernatant was replaced with 0.5 mL 0.1 M sodium bicarbonate. Sulfo-Cy5-NHS dye (Click Chemistry Tools 1076-100) was added at a molar ratio of 2:1 relative to amine PEG and rotated at 4° C. overnight. The particles were then washed 3 times in PBS+0.1% (w/v) Tween20, and 2 times in PBS. Particles were characterized by dynamic light scattering, TEM, and UV-Visible spectroscopy (FIG. 5).

### Silica Nanoparticles

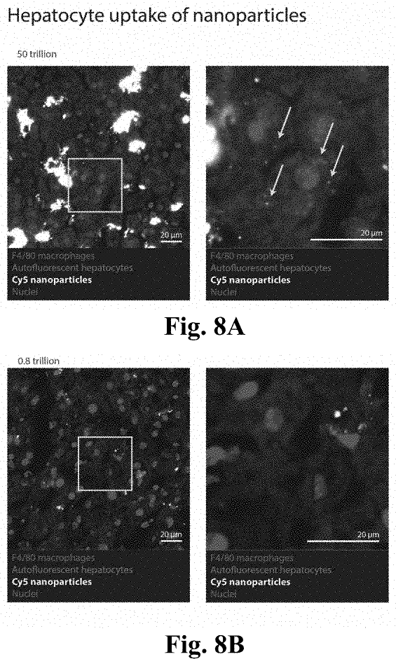

5 mg of 5 kDa ortho-pyridyl disulfide succinimidyl valerate OPSS-PEG-SVA (Laysan Bio) and 19.8 mg of 5 kDa SVA-mPEG (Laysan Bio) were weighed and dissolved in a total of volume of 250 μL of 10 mM HEPES+1 mM NaCI. This PEG solution was mixed with a 500 μL of 50 nm aminated silica nanoparticles (nanoComposix SIAN50-25M), vortexed and incubated at 60° C. for 1h. The solution was centrifuged at 10000g for 45 min and washed twice more with HEPES buffer (pH 7.44) buffer at 8500g for 45 min. 500 μL of 10 mM Tris-2-carboxyethyl phosphine (TCEP) in HEPES buffer was transferred to resuspend the pellet of SiNPs, and the solution was placed at 60° C. for 30 min. It was washed twice by centrifugation at 8000g in 500 μL HEPES. SiNPs were mixed with a 100 μg of sulfo-Cy5-maleimide at room temperature overnight. The next day, this was washed twice with HEPES+0.05% (w/v) Tween-20. Particles were characterized by dynamic light scattering, TEM, UV-Visible spectroscopy, and fluorimetry. (FIG. 8).

### Animal Models.

All animal research was reviewed by and conducted in accordance with the animal ethics committee from the Division of Comparative Medicine at the University of Toronto (protocols #20011909, #20012099, #20011605). 6 week old female BALB/c mice were purchased (Charles River Laboratory BALB/cAnNCrl). 4T1 cells were a generous gift from Dr. Reginald Gorczynski. 4T1 tumours were induced as described³ into 7-week old mice. Briefly, 1 million 4T1 cells between passages 3-20 in 100 μL of serum-free, antibiotic-free RPMI (Wisent Bioproducts 350-000-EL) were injected into the right inguinal 5^{th} mammary fat pad using a 25G needle. The needle was inserted 1 mm lateral and 3 mm caudal to the nipple, and advanced towards the nipple by 2 mm. Mice were injected with nanoparticles 2 weeks post-induction unless specified otherwise. We aimed to investigate smaller tumours to minimize confounding by the necrotic core and maximize quantification accuracy. Tumour growth was homogeneous (average 14-day tumour size and standard deviation: 0.38±0.15 g). Tumours were size-matched between treatment groups to enable %ID and %ID/g comparisons between groups. MMTV-PyMT transgenic mice on FVB/n background were purchased as a breeding pair (Jackson

Laboratories 002374). Offspring were sent for genotyping from 3mm tail snips using real time PCR at TransnetYX. Female heterozygous mice were injected with nanoparticles while they were between 10-13 weeks old (tumours ˜580±470 mm³). 6 week old CD1 nude mice were purchased (Charles River Laboratory Crl:CD1-Foxn1^{nu}). Cancer cells were injected orthotopically into the mammary glands as above. 8 million MDA-MB-231 cells between passages 2-5 in 200 μL of a mix of 50% (v/v) Dulbecco's Minimal Essential Medium (with 10% (v/v) fetal bovine serum and 1% (v/v) penicillin/streptomycin) and 50% (v/v) Matrigel (Fisher Scientific CB-40234), or 1 million 4T1 cells as above. Mice were injected with nanoparticles while they were between 12-14 weeks old (tumours ˜300 mm³). Transgenic BALB/c mice expressing EGFP on cells expressing CSF-1R (Csf1r-EGFP; also c-fms-EGFP) were bred from a generous breeder pair gift from Dr. Mikala Egeblad, and were first created by Dr. David Hume's lab⁴.

### Gold Nanoparticle Biodistribution.

Mice were euthanized by isoflurane overdose followed by cervical dislocation at 2, 8, 24, or 72 hours post nanoparticle injection. Mouse organs were collected and weighed into borosilicate tubes for analysis by ICPMS^{5,6}. Collected organs included: heart, lungs, liver, spleen, stomach (with diet), large intestines (with diet), small intestines (with diet), kidneys, uterus, dorsal skin, and cardiac blood. For endpoint cardiac blood, mice hearts were quickly exposed by dissecting through the thoracic cavity. A 25G needle/1 mL syringe was inserted into the right ventricle of the beating heart. ˜600 μL cardiac blood was drawn then transferred to a glass tube. For reference, a control tube containing a known proportion of the injected dose was also prepared in a borosilicate tube. 800 μL of 16 M nitric acid (Caledon 7525-1-29) was added to each sample. They were digested at 80° C. in a water bath for 1 hour, and then digested overnight at 50° C. The next morning, 200 μL of 12 M hydrochloric acid (Caledon 6025-1-29) was added and the samples were digested at 80° C. for ≥1 hour. Samples were collected into polypropylene 50 mL centrifuge tubes (Biomart 110708) and then diluted to 40 mL in deionized water for a final acid concentration of 2.5% (v/v). 10 mL of these samples were then filtered through a 0.22 μm PES filter (Fisher Scientific SLGP033RS) into 15 mL centrifuge tubes (Sarstedt 62.554.002). A standard curve for elemental gold and magnesium were prepared by dilution in 2% (v/v) nitric acid and 0.5% (v/v) hydrochloric acid, with concentrations ranging from 0.0001 to 100 mg/mL, with a reference blank of 0. All samples were then quantified using a NexION 350x ICP-MS (PerkinElmer) with mass analyzer set to magnesium Mg 24, iridium Ir 192, and gold Au 197. A 500 μL injection loop was used, and each sample was mixed with carrier solution (2% (v/v) nitric acid) and iridium internal standard (1 μg/mL) before injection into the analyzer. Percent injected dose was the measured gold mass of each sample divided by the measured gold mass of the injected dose.

### Blood Collection and Gold Quantification.

For blood half-life studies, mouse tail veins were punctured using a 29G needle and bled at various time points for repeated measurements of each mouse. 5-10 μL of blood was collected in capillary tubes (Fisher 2120-22260950) and transferred to 1.5 mL centrifuge tubes (Fisher 14222155). All blood was weighed and stored at 4° C. until further analysis. 200 μL of 16 M nitric acid and 50 μL of 12 M hydrochloric acid were added in sequence. These samples were then diluted into 9.75 mL of deionized water. Samples were not filtered to prevent gold adsorption and loss onto filter. Samples were analyzed by ICPMS as described above.

In vitro Saturation.

RAW264.7 cells were seeded into a 24-well non-tissue cultured treated plates (Falcon 351147) at a density of 136,000 cells in 333 μL of DMEM media. 50 nm gold nanoparticles (concentration range: 0.25-4 nM) with a 5 kDa methyl-PEG-SH surface density of 0.17 PEG/nm² were added to the wells immediately and cells were exposed for 24 hours. At the end of the experiment, wells were gently washed with calcium-free PBS three times. Then cells were removed from the wells by vigorous pipetting and pelleted in a 1.5 mL centrifuge tube. The supernatant was frozen, and the pellets were frozen at −20° C. until analysis. ICP-MS was performed as previously described⁶ to determine the amount of gold in each pellet. Pellets were digested with 800 μL of nitric acid and 200 μL hydrochloric acid, then number of cells was determined through magnesium concentration, compared to the magnesium concentration of a sample of a known number of cells. Gold mass per cell was converted into particles per cell using the density of gold (19 g/cm³).

### Intravital Microscopy Preparation

Csf1r-EGFP BALB/c mice were used between 8-11 weeks of age. Anesthesia in mice was induced with 5% isoflurane in humidified oxygen, and maintained at 2.5-3% isoflurane. Anesthetized mice were shaved on their abdomen and placed supine onto a warmed heat pad. A skin incision was made mid-abdomen and extended superiorly past the xyphoid process. Fascia between the skin and peritoneum was blunt-dissected. Excess fur was wiped away with sterile PBS-soaked cotton swabs. An incision was made just inferior to the xyphoid process in the peritoneum, and extended superiorly to mid-thorax. Longitudinal blood vessels in the peritoneum were cauterized before lateral incisions were made. The liver was exposed, gently pulled inferiorly with a sterile cotton swab to expose the falciform ligament. The falciform ligament was cut. The mouse was placed onto its right side, and the left liver lobe was gently pulled out of the abdominal cavity and onto a platform. Care was taken not to disturb the superior aspect of the lobe, which was to be imaged. A few drops of sterile PBS was applied onto the liver surface. A #1 glass slide (Fisherbrand 12544E) was gently placed onto the liver with care to not compress sinusoids. A drop of PBS was placed between a water-immersion lens and glass slide. Wide-field fluorescent imaged was done under the FITC filterset to confirm blood flow in sinusoids. A 29G needle loaded with injection solution was inserted into a lateral tail vein, to be injected after recording began.

### Intravital Microscopy Imaging

Microscopy was performed using a Zeiss LSM 710 confocal microscope on an upright AxioExaminer stand with a 20x/1.0 Numerical Aperture (NA) water immersion Plan Apochromat objective lens. EGFP, Cy3, and Cy5 were excited using 488, 561 nm, and 633 nm laser lines, and detected with emission windows 493 to 556 nm and 638 to 759 nm. Laser power was set between 2-10%, and gain was manually adjusted to maximize signal-to-noise ratio. The confocal pinhole size was set to 1-2 Airy units. Images were collected at a rate of 1 image every 3 seconds with a resolution of 512×512 pixels, averaging 2. Laser scan speed was set to 1.5 seconds/frame with bidirectional raster scanning. 2-5 minutes of video were pre-recorded before nanoparticles were injected to establish a baseline. Recording continued up to 45-60 minutes after injection.

### Intravital Microscopy Analysis

Images were processed using ImageJ/FIJI⁷ (National Institutes of Health). Macrophages were manually traced and masked from the EGFP channel. Nanoparticle quantification was quantified using this mask in the Cy3 or Cy5 channels at 2, 5, 10, 15, and 25 minutes post-injection. Breathing artifacts during imaging often created dark bands across the image; such dark spots were replaced using Adobe Photoshop 21.0.2 by cutting and pasting time-adjacent frames to form a complete cell image. Uptake rates were calculated as average slopes from the uptake vs time values.

### Silica Nanoparticle Quantification.

Mouse organs were imaged in a Kodak in vivo Multispectral Imaging System (Bruker Corporation). Excitation and emission filters of 650 nm and 700 nm were used with an exposure time of 10 minutes. ImageJ/FIJI (National Institutes of Health) was used to obtain the signal density of tumours and livers. The tumour/liver ratio was obtained by dividing the tumour signal density by the liver signal density of each mouse, and then obtaining an average of the ratios.

### Liver Histology

Sinusoidal wall analysis. 4T1 tumour-bearing mice were injected with one of two doses of nanoparticles (0.8 trillion and 50 trillion) through the tail vein. 15 minutes later, mice were euthanized. A midline abdominal incision was made to expose internal organs. Intestines were lifted out of the abdominal cavity to provide easier access to liver. The median lobe was excised and immersed in 10% neutral buffered formalin (Sigma HT501128). This lobe was fixed for 24 hours at room temperature, then transferred to 70% ethanol solution for storage. Tissues were processed by the The Centre for Phenogenomics (Toronto, Ontario, Canada). Briefly, the liver was routinely processed and embedded in paraffin wax. Tissue sections cut at 5 μm were collected onto charged slides and baked prior to immunohistochemical staining. Tissue sections were deparaffinised through xylenes and an alcohol gradient and taken to water. Antigen retrieval was performed using H.I.E.R. (Heat Induced Epitope Retrieval) with citrate buffer (pH 6) for 7 minutes. Non-specific antibody binding was blocked with Dako Protein block (Agilent X0909) for 10 minutes, followed by Armenian Hamster Anti-CD209b (ThermoFisher Scientific 14-2093-81) diluted 1:200 and incubated overnight at 4° C. After washes, Goat Anti-Armenian Hamster Alexafluor 488 (Abcam ab173003) secondary antibody was used to visualize CD209b positive staining. This was imaged using an Olympus VS120 whole slide imaging system at 20× magnification in the FITC (CD209) and darkfield (nanoparticles) channels. Images were visualized using ImageJ to identify vessels and nanoparticles. Nanoparticle distribution was performed using the Line tool drawn from one wall of a blood vessel to the other, and quantified using the “Line Profile” function. 20 blood vessels were randomly chosen per liver for a total quantification of 60 vessels per dose. The vessel widths were all expanded/normalized to the largest vessel width of the group, and the nanoparticle signals of smaller vessels were expanded to this size by interpolation using MATLAB's interp1 function (MathWorks MATLAB R2017b).

Liver cell analysis. 4T1 tumour-bearing were injected with doses of nanoparticles ranging from 50 billion to 50 trillion. Mice were sacrificed 24 hours later. The median lobe of the liver was dissected and cryopreserved in “optimum cutting temperature” compound (VWR 25608-930) and indirect contact with liquid nitrogen. Histological slides were processed at the The Centre for Phenogenomics. Briefly, 8 μm thick sections were sectioned on a Cryostar NX70 cryostat. The tissue was stained with anti-F4/80 (Abcam ab6640) at a 1:200 dilution for 1 hour at room temperature. This was imaged using an Olympus VS120 microscope at 20x magnification in the DAPI (nuclei), FITC (autofluorescence), TRITC (F4/80) and Cy5 (nanoparticles) channels. Images were visualized using ImageJ to identify macrophages (F4/80+), hepatocytes (autofluorescence+) and nanoparticles. Macrophages were gated manually on F4/80+ cells and hepatocytes were gated manually on F4/80⁻ autofluorescent+cells using imageJ. These gates were used as masks to quantify the average Cy5 nanoparticle intensity per cell.

### Modelling.

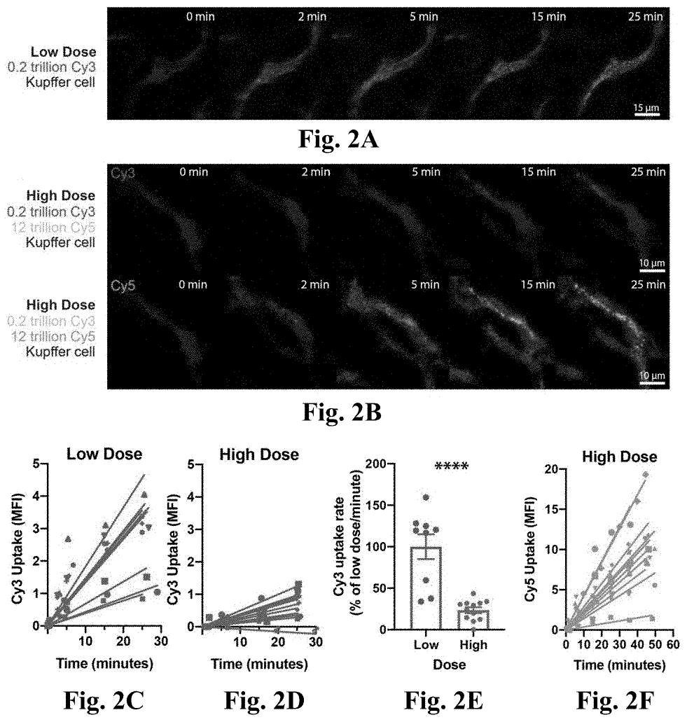

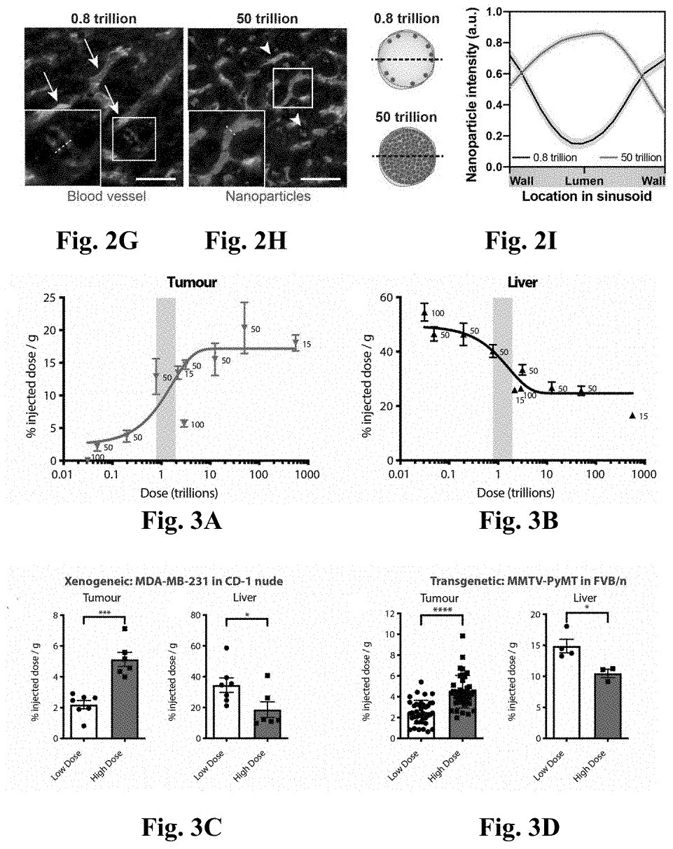

Kinetic accumulation in the liver and tumour were modelled using a compartment model composed of 4 compartments: blood, liver (Kupffer cell), liver (other cells), and other organs. The following assumptions were made: the blood compartment started at 100% and delivered nanoparticles to all other compartments, nanoparticles were always perfectly mixed in the blood (i.e. no local concentration differences), flow of nanoparticles from other compartments back into blood was negligible, flow of nanoparticles between non-blood compartments is negligible, and all compartments had a finite capacity. The compartment model, outlined in FIG. 2A, dictates the following equations:

NP+T⇄_{k-T}^{kT}NPT

NP+KC⇄k-KC^{kKC}NPKC

NP+OC⇄_{k-OC}^{kOC}NPOC

NP+O⇄_{k-O}^{kO}NPO

where NP is the concentration of total circulating nanoparticles and T, KC, OC, and O are the concentrations of remaining capacity in each of the compartments: Tumour, Kupffer Cells, Other Cells, Other organs. NPT, NPKC, NPOC, and NPO are the concentrations of nanoparticles in each of the organs. Each kₓ represents the rate of the transfer of nanoparticles from blood to the respective compartment, and k₋ₓ represents transfer of nanoparticles from organ back to blood (negligible). These rate equations can described by a system of ordinary differential equations:

$$\frac{dNP}{dt} = - {k}_{T} NP ( {T}_{0} - NPT ) - {k}_{KC} NP ( {KC}_{0} - NPKC ) - {k}_{OC} NP ( {OC}_{0} - NPOC ) - {k}_{0} NP ( {O}_{0} - NPO )$$

$$\frac{dNPT}{dt} = {k}_{T} NP ( {T}_{0} - NPT )$$

$$\frac{dNPKC}{dt} = {k}_{KC} NP ( {KC}_{0} - NPKC )$$

$$\frac{dNPOC}{dt} = {k}_{OC} NP ( {OC}_{0} - NPOC )$$

$$\frac{dNPO}{dt} = {k}_{O} NP ( {O}_{0} - NPO )$$

where T₀, KC₀, OC₀, O₀ are boundary conditions for weight-normalized capacities of the unbound tumour, Kupffer cell, other liver cell, and other tumour compartments. We limited KC₀ to be 1 trillion nanoparticles/gram. We set k values such that k_{KC}>k_{OC}>k_{O}>k_{T}. We used two doses, 5 trillion and 0.2 trillion. Using these equations and initial conditions, we simulated the evolution of nanoparticle concentrations in these compartments over 24 hours using MATLAB's ode23 function. To decrease computational time, we decreased the doses by 1e10 and the k constants accordingly.

### 3D Microscopy Preparation−Labelling.

Samples were prepared for and imaged in 3D microscopy as previously described^{8,9}. 5 minutes prior to euthanization, mice were injected with GSL-1-Cy3 (150 pg, Vector Labs, conjugated with 15 pg Cy3-NHS, click chemistry tools) to label blood vessels.

### 3D Microscopy Preparation—Perfusion Fixation.

Mice euthanized through perfusion fixation using 60 mL saline solution (phosphate buffered saline, 0.5% (w/v) sodium nitrite, 10 U/mL Heparin) and 80 mL monomer fixative solution (phosphate buffered saline, 2% (w/v) acrylamide, 4% (v/v) formaldehyde, 0.25% VA-044 initiator). Tissues were fixed for 7 days in monomer solution, then degassed, purged with argon and incubated at 37° C. for 3 hours to polymerize the acrylamide and convert the tissue into a hydrogel. Tissue blocks of 1 mm thickness were placed in clearing solution (4% (w/v) sodium dodecyl sulfate, 200 mM sodium borate, pH 8.5) for 10 days, transferred to borate solution (200 mM sodium borate, pH 8.5, 0.1% (w/v) TritonX-100, 0.01% (w/v) sodium azide) for 1 day, then stained with DAPI (400 μmol/mg of tissue) for 2 days, transferred back to borate solution for 1 day and then placed in TDE solution (67% 2,2′-thiodiethanol, 33% borate solution) for 1 day before imaging.

### 3D Microscopy.

Tissue blocks were imaged on a Zeiss Lightsheet Z.1 microscope using a 20X, NA 1.0, RI 1.45 clearing objective. Images were acquired at 0.7X zoom. Gold nanoparticles were visualized using dark-field imaging on the same microscope by removing the laser block and emission filters⁹. 3 stacks were acquired for each sample.

### 3D Microscopy—Image Analysis.

Image analysis. Images were imported into MATLAB using the Bio-Formats toolbox and downsampled in X and Y directions to achieve isotropic resolution in all dimensions. Blood vessels (GSL-1-Cy3) were segmented using a local threshold and distances from blood vessels was assessed using a Euclidean distance transformation. Nanoparticle intensity (darkfield scattering intensity) was normalized to the maximum intensity at the blood vessel wall in a given image. Normalized mean nanoparticle intensity was plotted as a function of distance from the nearest blood vessel.

### Tumour Disaggregation for Single Cell Analysis.

Tumours were collected and stored in 4° C. PBS until all tumours were dissected from mice. Tumours were manually diced with a razor blade into ≤1 mm³ pieces. This slurry was transferred into 5 mL of a digestion solution containing Hanks Balanced Salt Solution (HBSS; Gibco 14185052) with 400 μg/mL collagenase IV (Sigma C5138) and 20 μg/mL DNase I (Roche 10104159001), pH 7.4, and incubated for 45-60 minutes under gentle rotation at 37° C. Disaggregated cells were filtered through a 70 μm mesh strainer, then centrifuged at 500×g for 5 minutes. The supernatant was discarded, then the pellet was resuspended in 2 mL of RBC lysis buffer (BioLegend 420301) and incubated for 5 minutes. 13 mL of HBSS was added to the samples, and the sample was centrifuged at 500×g for 5 minutes. The supernatant was discarded, and the samples resuspended in 300 μL of blocking buffer containing HBSS, 0.5% (w/v) bovine serum albumin, and 2 mM EDTA (BioShop EDT001). Samples were diluted to 25 million cells/mL according to a standardized counter (Beckman Coulter ViCell XR using “default” cell type) and kept on ice.

Liver Perfusion and Disaggregation for Single Cell analysis.

The mouse was anesthetized using isoflurane (5% induction, 2% maintenance). A horizontal skin incision was made across the abdomen midline. Skin was retracted and the same incision was made onto the peritoneum to expose the viscera. Intestines were gently displaced outside to the left of the mouse to expose the portal vein and vena cava. A 21G needle connected to a peristaltic pump was inserted into the portal vein, the vena cava was cut open (to allow outflow), and perfusion was performed for 5 minutes at 5 mL/minute with a 10 U/mL heparin solution in 1× PBS (calcium-free). The liver was observed to turn in colour from red to brown. The solution was then exchanged for 3 mg/mL collagenase in HBSS at a flow rate of 5 mL/minute for 5 minutes to digest. The liver was observed to turn in colour from brown to tan/yellow. When the colour change was patchy, it indicated suboptimal perfusion; in these cases the 21G was poked into the liver and flow rate was reduced. At the end of digestion, the liver was carefully excised and placed into a solution of HBSS. The Glisson's capsule was cut and the liver was gently agitated with tweezers to dissociate hepatic cells into the solution. This cell solution was centrifuged at 25g for 2 minutes. The supernatant was labelled as non-parenchymal cells and the pellet was labelled as parenchymal cells. A portion of parenchymal cells were visualized under DIC microscopy to assess presence of hepatocytes; if no hepatocytes, we deemed the perfusion/digestion a failure and discarded the sample. Samples were diluted to 25 million cells/mL according to a standardized counter (Beckman Coulter ViCell XR using “default” cell type) and kept on ice.

### Flow Cytometry.

Stock antibodies were diluted 1/16 according to our antibody titration experiments (data not shown). All antibodies were purchased from BioLegend: anti-CD45-BV605 (clone 30-F11), anti-CD11b-BV711 (clone M1/70), anti-F4/80-AF488 (clone BM8), anti-Ly6G-PE/Cy7 (clone 1A8), anti-Ly6C-PerCP (clone HK1.4), anti-EpCAM-PE (clone G8.8), anti-CD31-BV421 (clone 390). Live/Dead staining was performed with Zombie NIR (BioLegend 423106) according to manufacturer's instructions. Antibodies were prepared in a v-bottom 96 well plate (Greiner M8185). FcyR blocking was done using anti-CD16/32 (BioLegend 101302) at a 1/1 dilution for 15 minutes on ice. Antibody staining was done for 30 minutes on ice. Two wash steps were performed at 500×g for 5 minutes using 200 μL of supernatant. Cells were fixed with 1.6% paraformaldehyde (Thermo Scientific 28906) in HBSS for 30 minutes, then washed once and stored up to 5 days at 4° C. before flow cytometry analysis. Events were acquired with a BD LSRFortessa X-20 (BD BioSciences). Gating was based on fluorescence-minus-one (FMO) controls. Compensation was performed using singly-stained OneComp beads (eBioscience 01-1111-42), ArC Amine Reactive Compensation beads (Invitrogen A-10346), and RAW264.7 cells with and without Cy5-tagged nanoparticles. Data were analyzed using FlowJo 10.0.7 (TreeStar Inc.).

### Transmission Electron Microscopy.

Samples for TEM were fixed in 2% (v/v) paraformaldehyde and 2.5% (v/v) glutaraldehyde in 0.1 M sodium cacodylate buffer, rinsed in buffer, post-fixed in 1% osmium tetroxide in buffer, dehydrated in a graded ethanol series followed by propylene oxide, and embedded in Quetol-Spurr resin. Sections 90 nm thick were cut on a Leica EM UC7 ultramicrotome, stained with uranyl acetate and lead citrate and viewed in an FEI Tecnai 20 TEM. Sample preparation and imaging was done at the Nanoscale Biomedical Imaging Facility (SickKids, Toronto, Canada).

### Characterization of Adsorbed Proteins on Gold Nanoparticles

All nanoparticle numbers were increased compared to in vivo doses to be able to extract enough protein for analysis. 1.2 trillion PEGylated gold nanoparticles in 100 μL PBS were added to 1 mL of pooled mouse serum (Sigma Aldrich M5905) and incubated at 37° C. for one hour. To isolate these protein-coated nanoparticles, the solution was centrifuged at 1,600 g for 30 minutes to pellet. Gold nanoparticles were resuspended in 950 μL PBS with 0.02% Tween20. This washing process was repeated three more times. The protein corona on liposomes was prepared similarly.

Protein coated gold nanoparticles (40 μL) were transferred to a new tube for further purification. 20 μL of sodium dodecyl sulfate (SDS) and 20 μL of 200 mM dithiothreitol (DTT) were then added and incubated at 80° C. for 10 minutes. The samples were then centrifuged at 18,000 g for 15 minutes, and 60 μL of the supernatant with extracted proteins were transferred to a new tube. The extracted proteins were then purified through acetone precipitation as described¹⁴. Briefly, 950 μL of trichloroacetic acid (TCA) (10% w/v in acetone) was added to each sample and incubated at −80 ° C. overnight. The samples were then centrifuged at 18,000 g at room temperature for 15 minutes, and the supernatant was discarded. 500 μL of sodium deoxycholic acid (SDC) was added to the pellet and vortexed thoroughly. Next, 100 μL of trichloroacetic acid (72% w/v in water) was added, and samples were left on ice for 2 hours. The samples were then centrifuged at 15,000 g for 15 minutes. The supernatant was discarded, and 950 μL of acetone was added. The samples were left at −80 ° C. overnight and centrifuged at 18,000 g for 15 minutes. The supernatant was discarded, and the samples were left to air dry.

Isolated and purified protein corona on AuNPs were then processed for characterization by liquid chromatography-tandem mass spectrometry (LC-MS/MS). 45 μL of 100 mM ammonium bicarbonate (NH4HCO3) and 5 μL of acetonitrile (ACN) was first added to samples. Additional 5 μL of 100 mM DTT was added. The samples were incubated at 37° C. for 60 minutes. 5 μL of an alkylating agent iodoacetamide (500 mM) in 100 mM NH₄HCO₃ was added. The solution was kept in the dark for one hour. This was followed by 2 μL of 0.25 μg/μL trypsin solution to digest the peptides. The digestion was stopped by adding 5 μL of 20% v/v formic acid. The proteomic analysis of these processed peptides is carried under the same steps and instrumentation settings as described previously¹⁵. Briefly, peptides were desalted on a C18 LC column before applying to the column. The elution takes place over one hour at a flow rate of 250 nL/min under 0 to 35% ACN gradient. Peptides were analyzed on a linear ion trap-Orbitrap hybrid analyzer, LTQ-Orbitrap Elite hybrid mass spectrometer (ThermoFisher). Spectral counts of each protein were then analyzed in Scaffold (Proteome Software).

### Statistics.

Statistical analysis was performed using GraphPad Prism 8. Multiple linear regression for the meta-analysis was conducted with SPSS Statistics (v21.0.0.0). Each figure caption describes the statistical test used and the statistical significance convention. Two-tailed unpaired t-tests were used to compare the means of two independent samples against each other. Two-way ANOVAs were used to compare the influence of two independent variables on a continuous dependent variable, and if the two independent variables have synergistic interactions. The Bonferroni adjustment corrects for multiple post-hoc analyses, as more tests results in more chances a type I error would be made. We chose Bonferroni because it is the most stringent adjustment (p=α/n, where α=0.05 and n is the number of post-hoc tests used) and therefore maximizes the robustness of conclusions from our results. All groups in t-tests and ANOVAs were assumed to be normally distributed and with equal standard deviation between groups. Statistical significance was determined when P<α, where α=0.05.

### Results

High Doses Overwhelm Liver clearance and increase half-life of nanoparticles

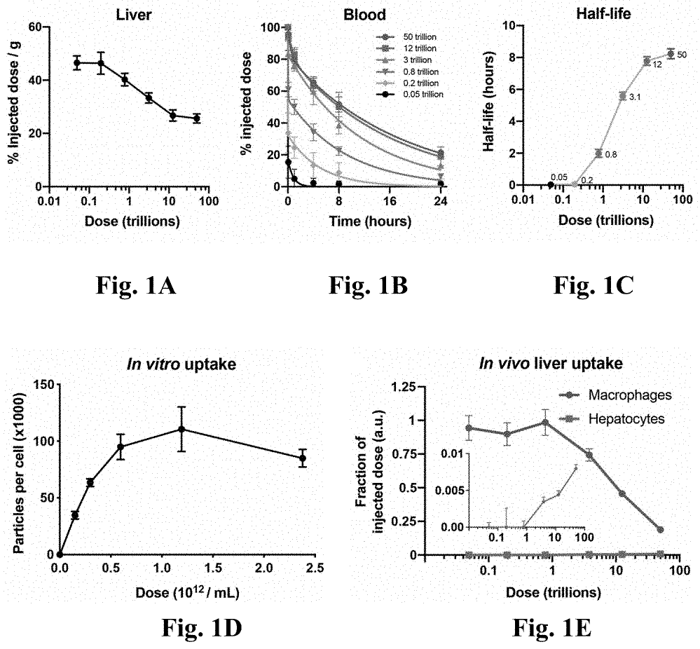

We began by investigating dose-dependent liver clearance of nanoparticles. We injected 4T1 tumour-bearing BALB/c mice i. v. with varying doses of 50 nm PEGylated gold nanoparticles (FIG. 5) that spanned from 50 billion to 50 trillion nanoparticles. We chose gold nanoparticles because they are inert and can be detected over a broad range of doses with high sensitivity. We used tumour-bearing mice because tumour presence alters clearance of nanoparticles²⁹. We sacrificed the mice 24 hours after injection and quantified gold biodistribution using inductively coupled plasma-mass spectrometry. We observed that the liver accumulated a decreased proportion of injected nanoparticles as the dose increased (FIG. 1A). This corresponded to an increase in nanoparticle blood half-life from 2 minutes to 8 hours (FIGS. 1B-1C). This confirmed our hypothesis that liver clearance efficiency of PEGylated nanoparticles was dose-dependent and limited at higher doses.

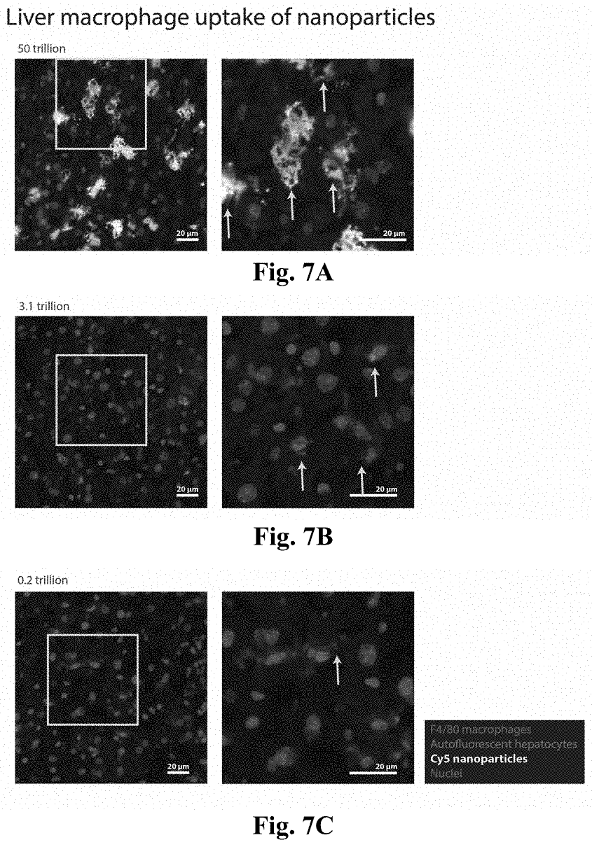

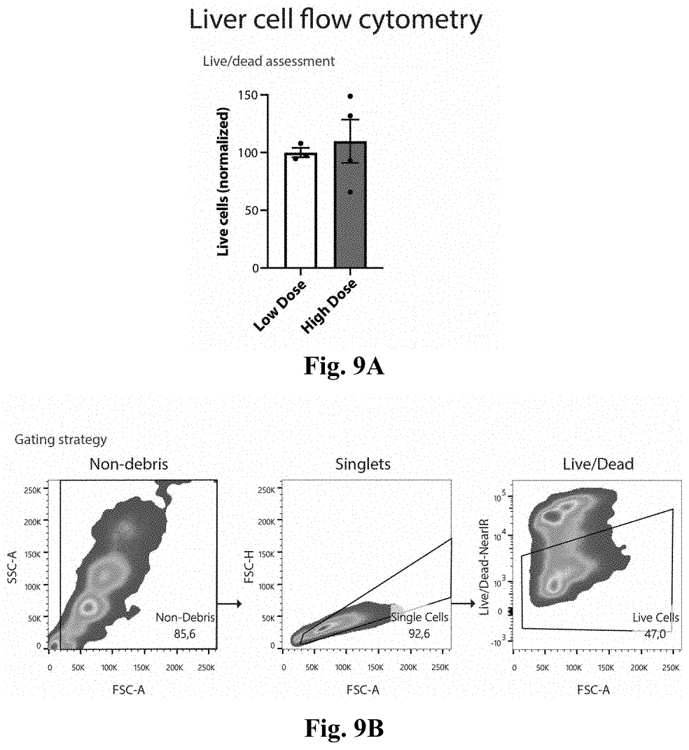

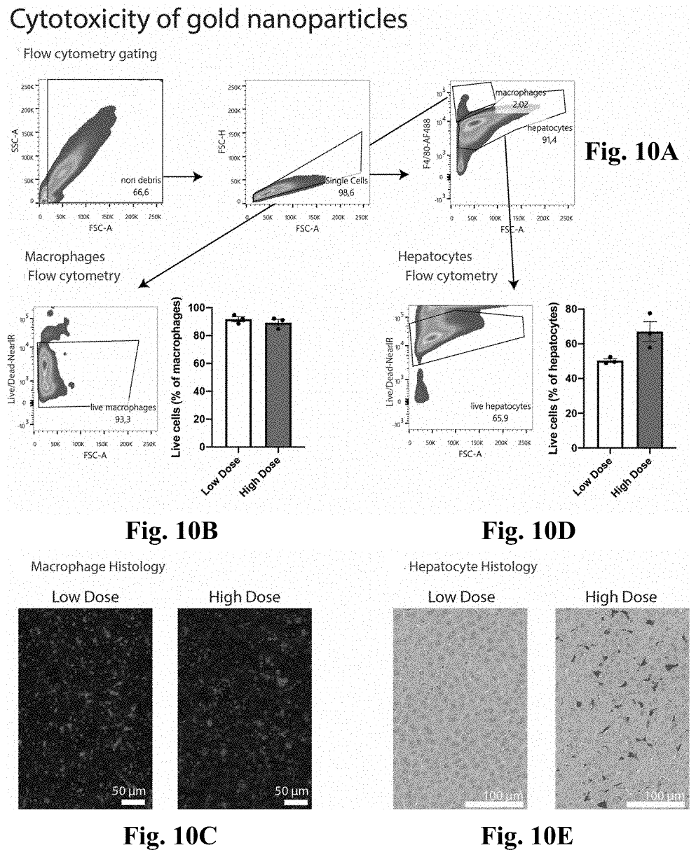

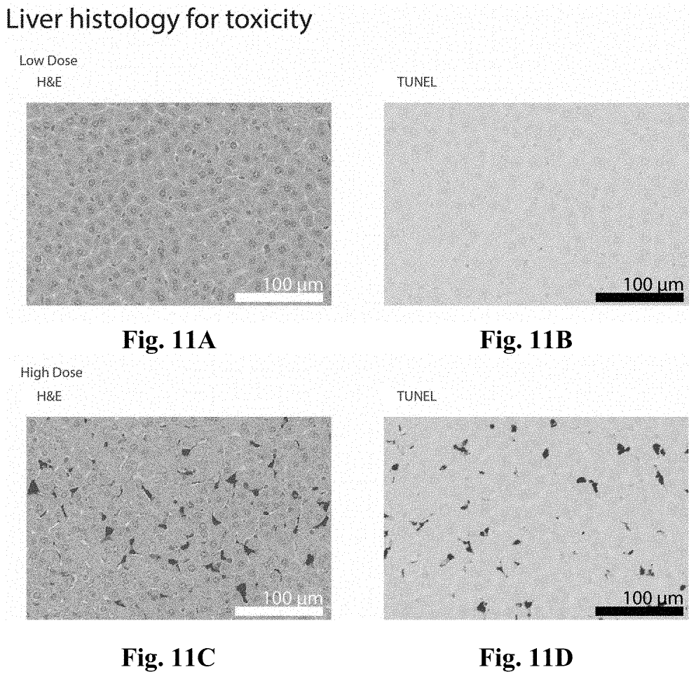

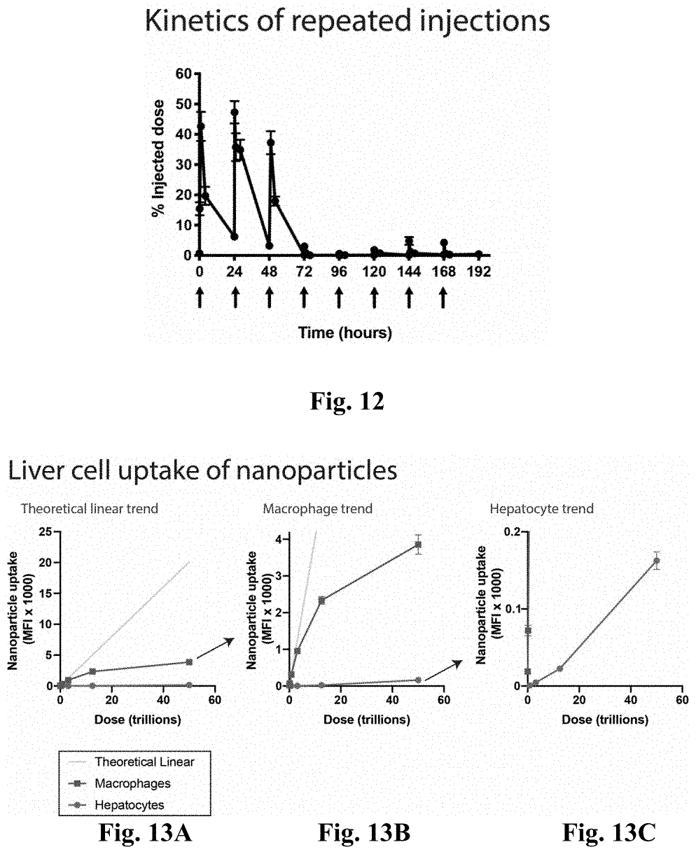

We investigated how much liver cells could take up over 24 hours, a commonly studied timepoint in nanoparticle studies. Liver macrophage cells internalize the most nanoparticles so we hypothesized that these cells were being overwhelmed. First, we incubated a macrophage cell line in vitro with gold nanoparticles of different doses and observed they could accumulate approximately 100,000 nanoparticles per cell in 24 hours, or approximately a maximum rate of 1 nanoparticle per second per cell (FIG. 1D). We tested if this limit correlated to Kupffer cells in vivo. Given that mice have on the order of 10 million Kupffer cells^{39,31}, we hypothesized that the total clearance rate limit of all Kupffer cells in a mouse liver was on the order of 1 trillion nanoparticles in 24 hours. We tested this hypothesis by analyzing livers 24 hours after nanoparticle administration using histology. We observed that Kupffer cells took up disproportionately fewer nanoparticles as dose increased past a threshold single dose of 1 trillion nanoparticles (FIG. 1E, FIG. 7). At doses above the threshold, a minor proportion of nanoparticles accumulated in hepatocytes (FIG. 1E, FIG. 8), suggesting hepatocytes served as a small liver accumulation reservoir once Kupffer cells were overwhelmed, as previously reported^{4,32}. We considered toxicity and opsonin-mediated clearance as potential mechanisms. This threshold was not due to Kupffer cell death as we did not observe liver cytotoxicity in the range of doses studied (FIGS. 9-11). Also, this threshold was not due to accelerated blood clearance (ABC) phenomenon because the ABC phenomenon takes 3-5 days to develop³³⁻³⁷ and the majority of our single doses already cleared after 2 days (specifically, it could develop in our PEGylated gold nanoparticle system after 4 days; FIG. 12). These results suggested that Kupffer cell uptake was fundamentally limited at single doses beyond 1 trillion nanoparticles in 24 hours.

Kupffer Cells Uptake Rates are Overwhelmed without Overwhelming Uptake Capacity

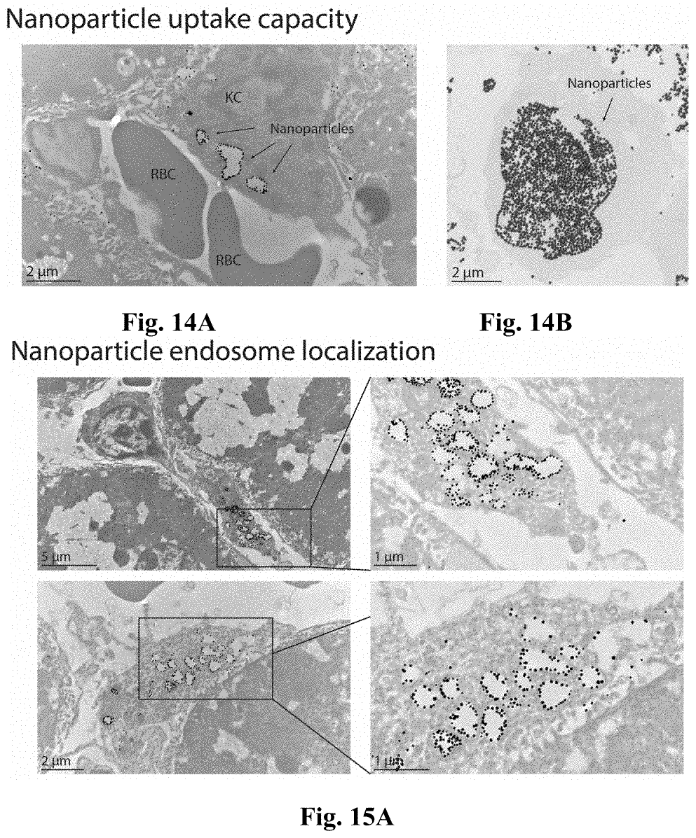

We investigated the mechanism for dose-dependent clearance. We hypothesized that high doses were overwhelming uptake rates because we did not observe Kupffer cells to saturate in total uptake capacity (FIG. 13). We used intravital microscopy to measure real-time nanoparticle uptake rates of in vivo Kupffer cells in the first 30 minutes of injection. We used Csf1r-EGFP mice³⁸ to identify Kupffer cells as GFP+ cells. We compared the Kupffer cell uptake rates of mice receiving a low dose with mice receiving a high dose. The low dose mice received 0.2 trillion Cy3-labelled nanoparticles only (FIG. 2A). The high dose mice received 0.2 trillion Cy3-labelled nanoparticles supplemented with 12 trillion Cy5-labelled nanoparticles (FIG. 2B). The nanoparticles were labelled with different dyes to discriminate uptake rate versus uptake capacity. Imaging in the Cy3 channel revealed that Kupffer cell absolute uptake of Cy3 nanoparticles was slower in mice that received a high dose than mice that received a low dose (FIGS. 2C-2E). This demonstrated that the higher doses inhibited uptake rates as more particles competed for uptake. We concurrently imaged these same cells in the Cy5 channel and observed continued uptake of Cy5 nanoparticles (FIGS. 2B&2F). TEM showed that uptake into Kupffer cells at 30 minutes was minor compared to the amount of uptake at longer times (FIG. 14). These conclusively showed that macrophage uptake capacity was not saturated. This is in direct contrast to studies employing the RES blockade, which assume capacity must be saturated by a pre-injection dose to reduce clearance of a second dose of therapeutic nanoparticles. These results proved that high doses of nanoparticles overwhelmed liver clearance because they overwhelmed Kupffer cell uptake rates and not uptake capacities.

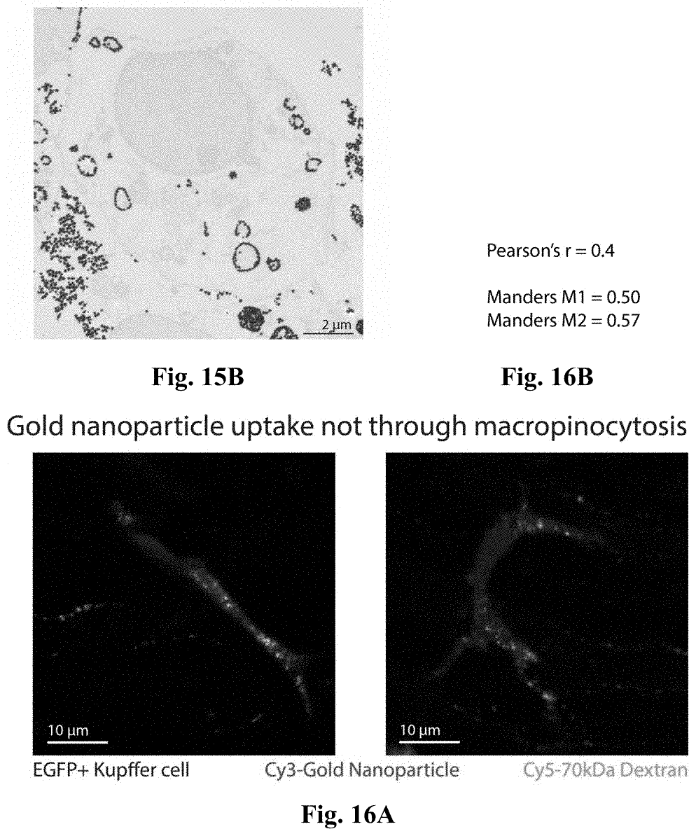

We next investigated why higher doses saturated uptake rates. There are three pathways of uptake in Kupffer cells: clathrin- and caveolin-mediated endocytosis, macropinocytosis, and receptor-mediated phagocytosis. We ruled out caveolae or clathrin-mediated endocytosis processes because these form 50-120 nm vesicles that do not take up particles larger than 35 nm³⁹, and we did not observe single nanoparticles enveloped in such endosomes on TEM (FIG. 15). We also ruled out dose-independent macropinocytosis because Cy3-labelled gold nanoparticles had poor intracellular localization with Cy5-labelled 70 kDa dextran, an established marker of macropinocytosis^{40,41} (FIG. 16). We hypothesized that Kupffer cells were taking up nanoparticles via receptor-mediated phagocytosis. TEM imaging of Kupffer cells 30 minutes after injection revealed that nanoparticles lined endosomal membranes but were absent from their luminal centres (FIG. 15), suggesting that only membrane-bound nanoparticles were internalized. Notably, nanoparticles crowded most of the endosome membranes. This suggested that most available binding sites had been occupied and that higher doses could not bind and be internalized. These patterns supported receptor-mediated phagocytosis. Since protein coronas influence nanoparticle uptake⁴², the adsorbed proteins on nanoparticles likely bound to Kupffer cell receptors. Further investigations using darkfield histology 15 minutes after injection revealed that the lower dose of nanoparticles sparsely outlined sinusoidal walls while the higher dose distributed centrally into the lumen. This further supported the hypothesis that higher doses overwhelmed receptors and binding sites on the walls of sinusoids. Since contact between a nanoparticle with a Kupffer cell or liver sinusoidal endothelial cell is necessary to clear the nanoparticle from circulation^{43,44}, the excess unbound nanoparticles in the center of the lumen had a lower probability to interact with cells and therefore had a higher probability of exiting the sinusoid to continue circulating. We conclude that high doses overwhelmed available receptors and binding sites on Kupffer cells, which limited the uptake rates of Kupffer cells, reduced liver clearance, and prolonged circulation.

### Modelling High Dose Uptake Kinetics

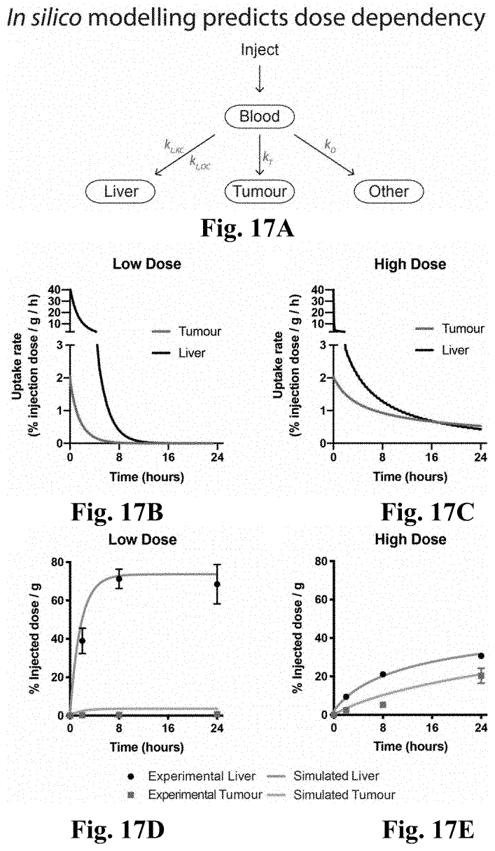

Next we investigated how overwhelming liver uptake rates would affect delivery to the tumour. We created an in silico compartment model to observe pharmacokinetics of nanoparticle transport from the blood to the liver and tumour (FIG. 17A). We inputted two doses: one below the 1 trillion/24 hours threshold (0.2 trillion) and one above (50 trillion). The outputs were nanoparticle uptake rates in the liver and tumour. Our simulation showed that in the low dose, the uptake rate of the liver was always faster than that of the tumour (FIG. 17B). In the high dose, the liver's uptake rate was proportionally less than in the low dose condition. It slowed rapidly and by 16 hours post injection, the high dose liver's uptake rate was slower than the tumour's (FIG. 17C). Mathematically integrating these uptake rates over the 24 hours of circulation yielded total accumulation over time. This showed that the high dose accumulated relatively less in the liver and delivered more to the tumour (FIGS. 17D-17E). We experimentally validated our compartment model using 4T1 tumour-bearing BALB/c mice injected with 0.2 or 50 trillion nanoparticles, and saw that the experimental results at 2, 8, and 24 hours post-injection aligned with the modelling results (FIGS. 17D-17E).

### High Doses Increase Tumour Delivery

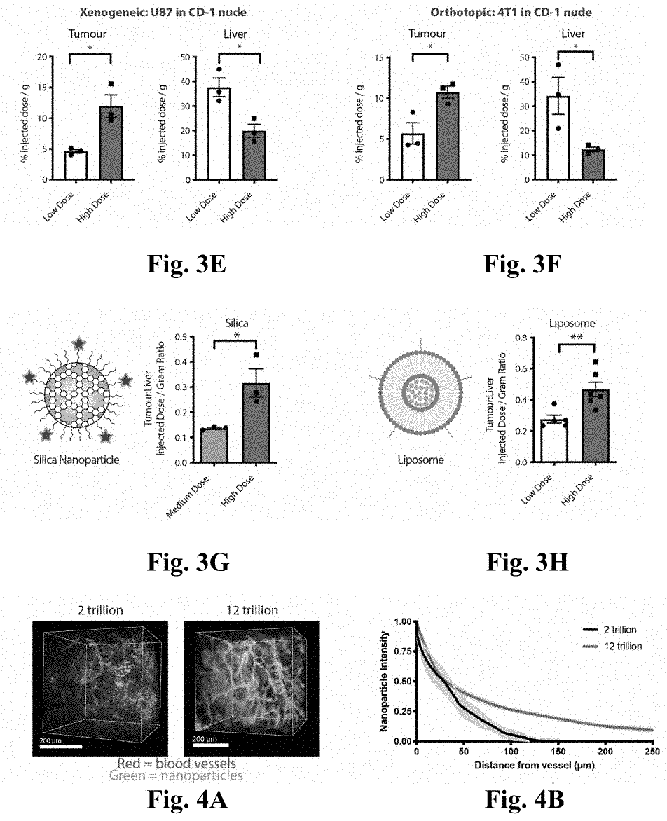

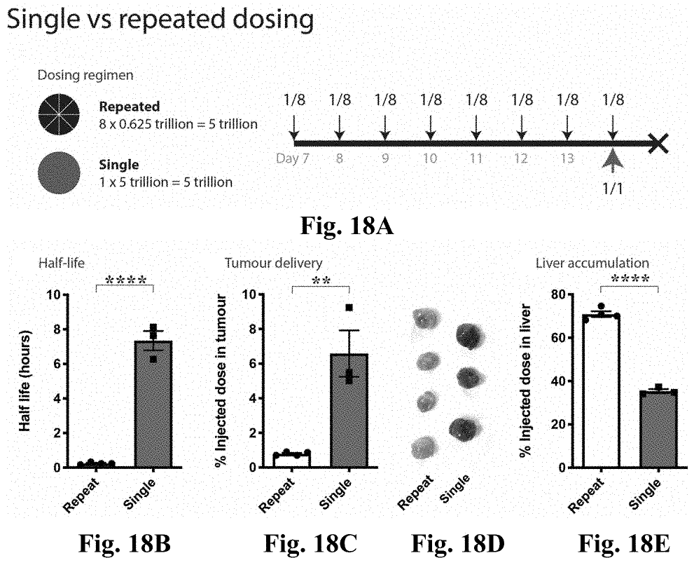

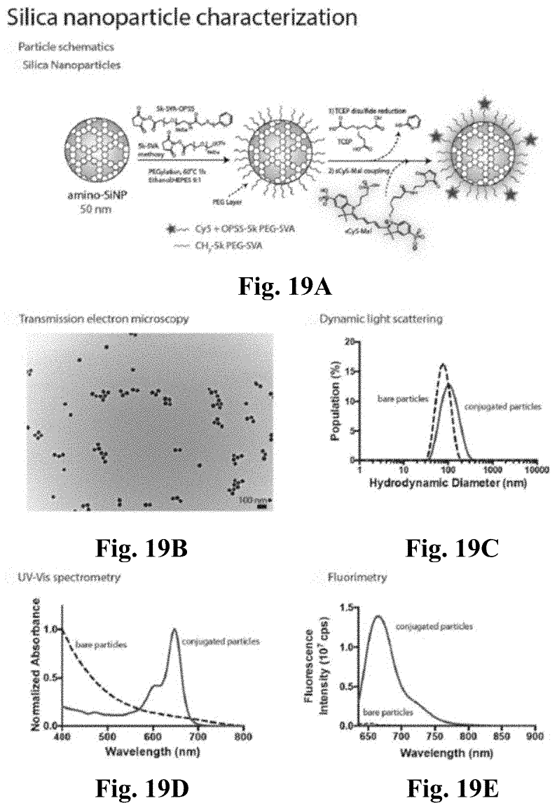

We further investigated tumour delivery as a function of dose to find a threshold and characterize delivery beyond such a threshold. We injected 4T1 tumour-bearing BALB/c mice with gold nanoparticles of various sizes, spanning doses from 30 billion to 550 trillion (3×10¹° to 5.5×10¹⁴) nanoparticles (FIG. 5). We confirmed experimentally in vivo that as dose increased, tumour delivery increased (FIG. 3A) and liver accumulation correspondingly decreased (FIG. 3B). Around 1 trillion nanoparticles marked the inflection points of the beginning of this nonlinear dose dependence for both organs, supporting our results that this was the minimal threshold dose that would begin to overwhelm the liver's clearance capacity and increase tumour delivery. We also compared the biodistribution of a single dose above this threshold against the same dose divided into multiple smaller doses below this threshold. We found that although the total dose was the same in both groups, the large single dose showed reduced liver accumulation, increased circulation half-life, and increased tumour delivery (FIG. 18), consistent with high doses overwhelming the uptake rate and not the liver's absolute uptake capacity. Together, these results confirmed that single doses exceeding a threshold of 1 trillion nanoparticles improved tumour delivery.

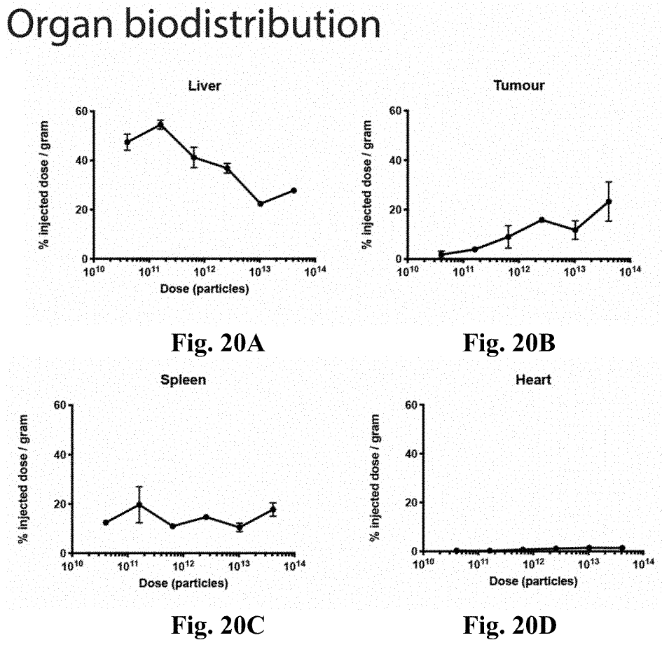

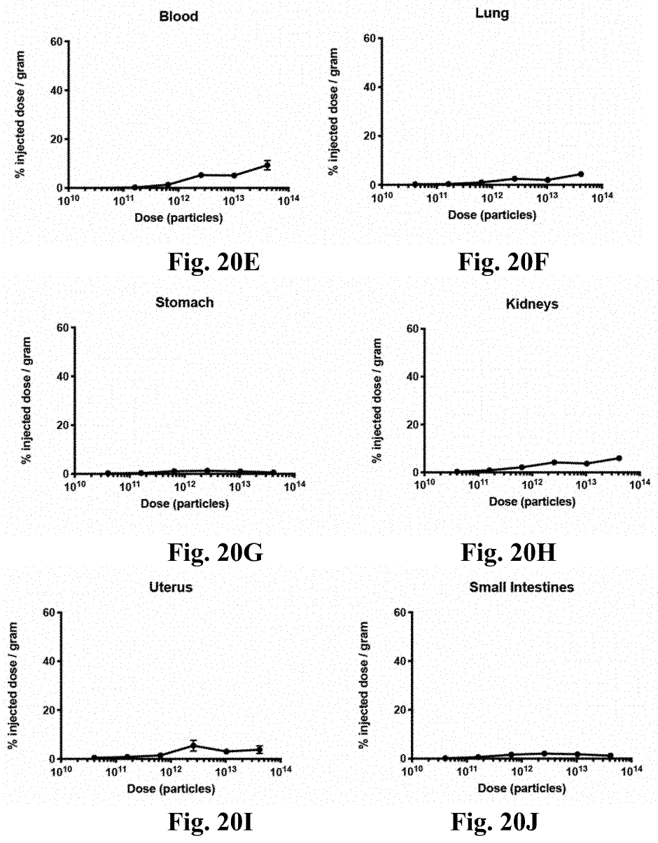

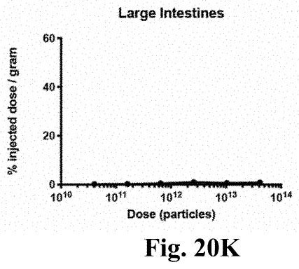

Beyond 1 trillion nanoparticles, tumour delivery efficiency continued to increase. At the highest dose, the liver and tumour accumulated the same amount of nanoparticles, gram-for-gram (16% injected dose/gram). Tumour delivery efficiency of the lowest dose was 0.03% of the injected dose. Tumour delivery efficiency of the highest dose was 12% of the injected dose, more than an order of magnitude improvement over the field's median 0.7%¹. This corresponded to a 7,300,000 times increase in absolute nanoparticle accumulation for only 18,000 times increase in dose. In contrast, the absolute accumulation in the liver increased only 6000 times for the same dose increase. We observed that this increase in tumour delivery efficiency at high doses was generally true for different mouse tumour models (xenogeneic, orthotopic, and transgenetic, FIGS. 3C-3H) and nanoparticle compositions (silica and liposome, FIGS. 3G-3H). Nanoparticle accumulation in other organs, including the spleen, displayed no or minimal dose-dependency, suggesting this was unique to the liver and tumour at this magnitude (FIG. 20).

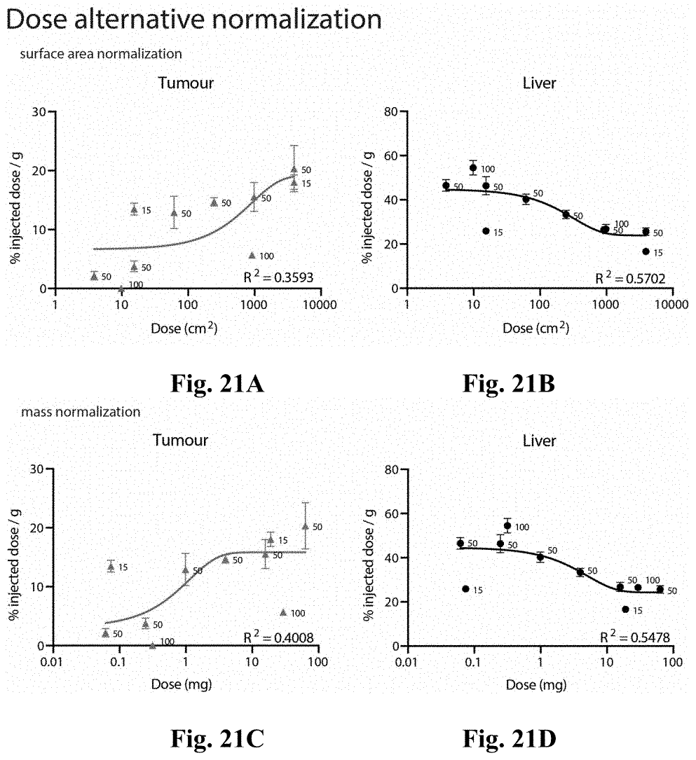

Interpreting dose data from other nanoparticle studies can be challenging. In typical nanoparticle size-dependency studies, the doses are normalized to surface area or mass, which results in higher numbers of smaller nanoparticles. Here when we normalized dose by units of surface area or mass, the correlation to tumour delivery dropped significantly and size-dependent accumulation in the tumour and liver emerged (FIG. 21). This suggested that nanoparticle dose may be a confounding variable in size-dependency studies, and that dose by nanoparticle number is a more robust parameter than size. Numbers of nanoparticles is also more biologically relevant than surface area or mass since the dose effectively corresponds to the numbers of receptors that bind nanoparticles. Combined, these highlight number of nanoparticles as the most relevant dose metric.

Overall, these results demonstrated that doses exceeding 1 trillion nanoparticles significantly increased tumour delivery for various nanoparticle sizes, compositions, and tumour models.

### High Doses Increase Nanoparticle Penetration and Delivery to Cells in Tumours

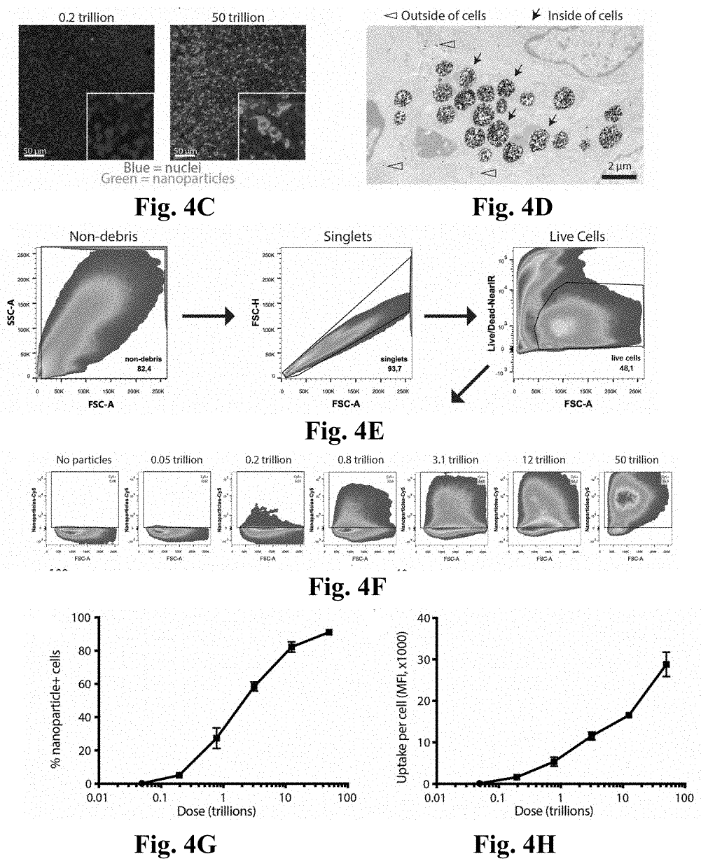

Next, we investigated how dose affected distribution of the increased nanoparticles delivered into the tumour. Generally, a major limitation to cancer drug delivery has been their incomplete cellular delivery. One reason is due to the difficulties of drugs diffusing across large distances⁴⁵. Nanoparticles face similar challenges that may be further exacerbated by their larger sizes⁴⁶. Since we observed exponentially larger increases in tumour accumulation at higher doses, we hypothesized that the increased concentration gradients would facilitate deeper nanoparticle penetration to more cells in the tumour. We conducted 3D CLARITY imaging to image nanoparticles near blood vessels^{47,48} and observed that higher doses of nanoparticles beyond the trillion threshold distributed further from blood vessels and deeper into the tumour tissues (FIGS. 4A-4B). High doses were well-distributed throughout tumour cells (FIG. 4C) and mostly intracellular (FIG. 4D). Quantification with flow cytometry showed that 93% of live cells had taken up nanoparticles at a high dose (FIGS. 4E-4G) and that each cell had taken up more nanoparticles (FIGS. 4E, 4F and 4H). In contrast, a sub-threshold low dose of nanoparticles (0.2 trillion) were sparsely distributed through the tumour tissue and only 0.7% of cells had internalized them (FIGS. 4A and 4C-4F). These results showed that high doses of nanoparticles enhanced their tumour penetration and improved delivery to the tumour cell population.

Artificially Enhancing does to Improve Drug Efficacy

To untangle the effects of nanoparticle number dose versus drug dose, we opted to artificially increase the nanoparticle dose using a benign filler nanoparticle to increase nanoparticle number dose only without increasing cytotoxic drug. We synthesize a benign filler gold, silica micelles or polymeric nanoparticle to be identical to a nanoparticle carrying the cytotoxic drug, but without loading any cytotoxic drug. We henceforth refer to them as “delivery enhancers” or “decoy nanoparticles” because their only function was to artificially increase nanoparticle dose to increase tumour delivery.

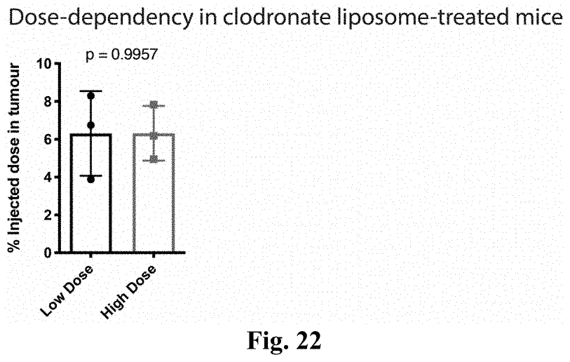

We designed a proof-of-concept efficacy experiment comparing two 4T1 tumour-bearing mouse groups that received the same dose of a therapeutic nanoparticle (corresponding gold nanoparticle, silica nanoparticle, micelles or polymeric nanoparticle), but with the experimental group receiving an additional dose of delivery enhancers. Both groups receive over 1 trillion nanoparticles to ensure significant dose-dependent delivery. The control group is injected with 4.6 trillion therapeutic nanoparticles (i.e. nanoparticles carrying the cytotoxic drug), and the experimental group was co-injected with an additional 45 trillion delivery enhancers (50 trillion nanoparticles in total). Although both groups receive the same amount of active drug, we expect that the experimental group will display greater efficacy because the delivery enhancers would improve tumour delivery of all nanoparticles, including the therapeutic ones. Simply increasing nanoparticle number could improve delivery efficiency and therapeutic efficacy. Negative control mice given only delivery enhancers will see no difference in efficacy compared to mice given a sugar water control, indicating these delivery enhancers had no antitumour efficacy on their own. We tested depleting Kupffer cells with clodronate liposomes before injecting high and low doses of gold nanoparticles. We observed that mice without Kupffer cells exhibited reduced dose-dependent gold nanoparticle tumour delivery (FIG. 22). Overall our results demonstrate that the number of nanoparticles administered directly impacts therapeutic efficacy.

### Delivery Enhancers are Superior to RES Blockade

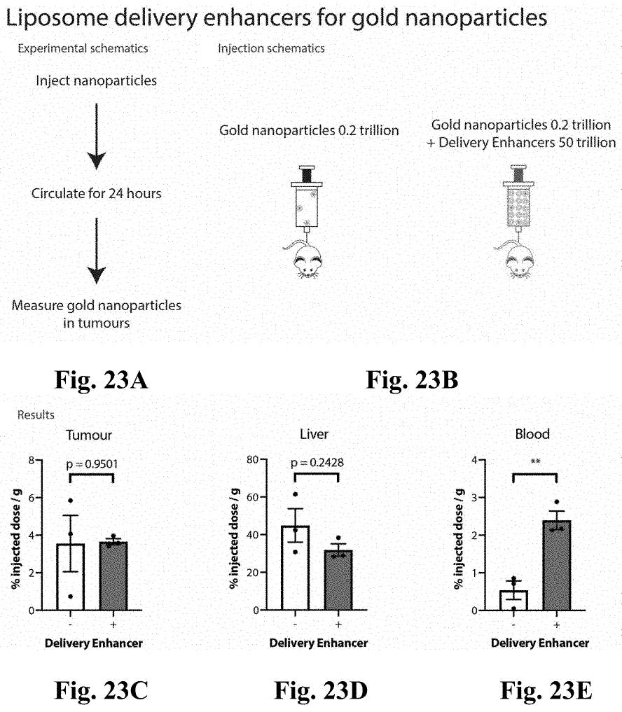

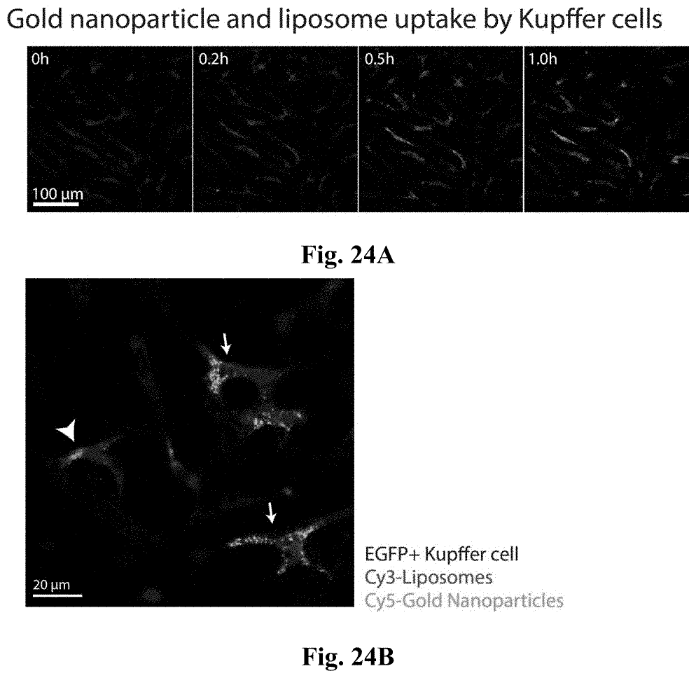

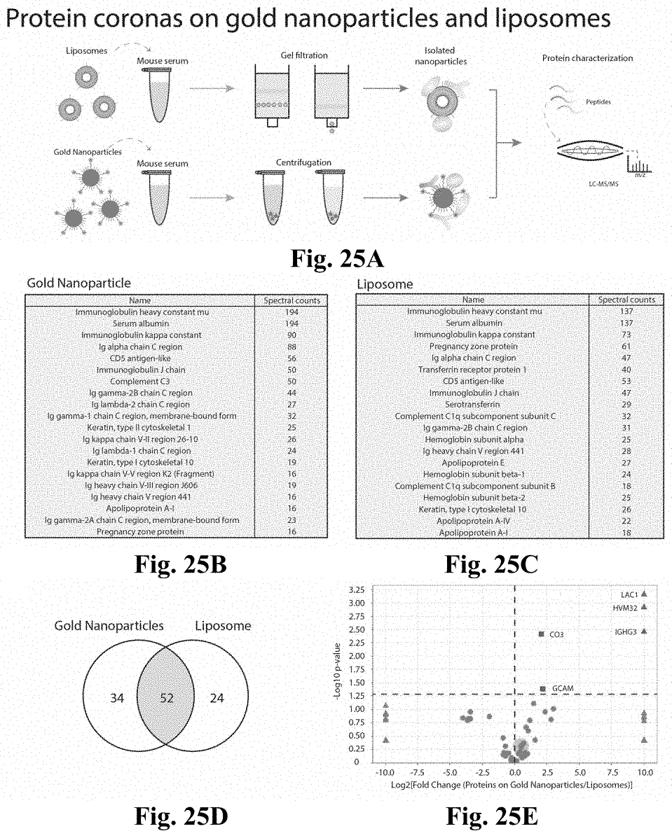

We studied if the decoy was a different material. Since clearance was receptor-mediated, and different nanoparticles have different protein coronas⁴², we hypothesized that different materials as decoys would not improve the pharmacokinetics and delivery of a material of interest⁵⁶. Indeed, we observed that 50 trillion co-injected liposomes could not increase tumour delivery of 0.2 trillion gold nanoparticles (FIG. 23). Intravital imaging confirmed that they were taken up by different Kupffer cells (FIG. 24) and LC-MS-MS confirmed that they had different protein coronas (FIG. 25). These results further support that receptors and binding sites on Kupffer cells must be fully occupied to slow their uptake rate. Lastly, as we determined, optimal doses should exceed 1 trillion nanoparticles to achieve this occupancy to reduce liver clearance and improve tumour delivery. These results demonstrate that delivery enhancers utilize the uptake rate mechanism to provide a superior solution to the RES blockade's challenges.

The 1 Trillion Threshold Exists throughout 10 Years of Literature

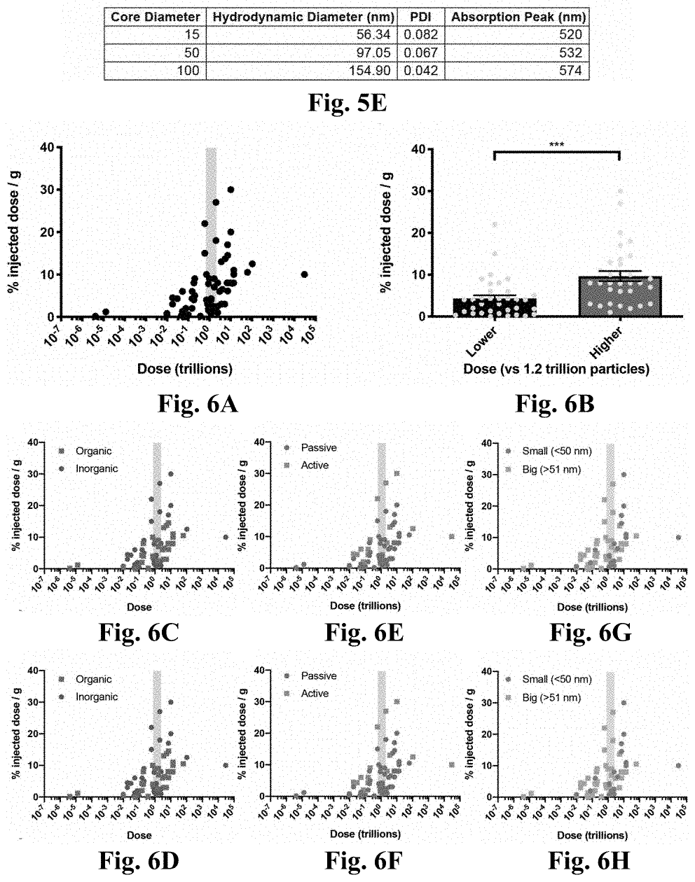

Finally, we sought to investigate the relevance of our conclusions to the rest of the nanoparticle delivery field. In 2016 our group performed a meta-analysis of the field's delivery efforts and analyzed all known parameters of nanoparticle design, including material, size, shape, and surface chemistry, as well as differences in tumour models¹. We did not include dose then. At that time, dose was not an obvious parameter that affected tumour delivery efficiency because it was not (and is still not yet) emphasized nor standardized in the literature. Here, we reanalyzed the set of papers to investigate nanoparticle dose as a parameter. We successfully recalculated nanoparticle doses by particle number in 40 publications and obtained 67 total data points of dose and delivery efficiency (Tables 1, 2). Using these calculations, we observed that dose by nanoparticle number correlated with delivery efficacy (FIG. 6A). We found that the median dose used in these papers was 1.2 trillion nanoparticles, just above the 1 trillion threshold observed in our experiments. Doses higher than the median dose had significantly higher delivery efficiency than doses below (FIG. 6B). We further stratified these data into inorganic vs organic, passive vs active, or small (<50 nm) vs large (>50 nm) and observed that this dose dependency and threshold persisted for all subdivisions (FIGS. 6C-6H). We also performed a multiple regression to predict tumour delivery efficiency from dose, material type, inorganic vs organic, targeting type, size (big vs small), tumour model, cancer type, and specific cell line. This regression statistically significantly predicted tumour delivery efficiency (F(8,55)=7.036, p=0.000002; Table 3a). Dose was the most significant variable in this regression (p=0.00002). This suggests that dose by nanoparticle number is the most important variable in determining nanoparticle delivery—more important than size, targeting design, nanoparticle type, or cancer model (Table 3b). The importance of dose by nanoparticle number was clearly unknown in these papers as the even distribution of nanoparticle doses above and below the trillion threshold suggested that doses were chosen arbitrarily. Indeed, none of these papers rationalized their choice of dose, and only 1 out of 117 papers reported investigating more than one dose. These results emphasize that the relationship between nanoparticle dose by number and tumour delivery is significant but so far, unrealized. They question if nanoparticles should instead be designed to minimize the drug loading per nanoparticle in an effort to maximize number of nanoparticles. These results are also especially important for studies that employ tracer doses of measurable as a proxy for pharmacokinetics distribution (i.e. radiolabelled), since our results show that the liver uptake kinetics and subsequent biodistribution of these low doses are different. We thus urge that all nanomedicines be rigorously evaluated for dose and use number of particles as a standard metric unit to maximize their potential.

TABLE 1
Studies from Wilhelm et al. Listed are the recalculated doses and the reported tumour delivery, in % injected dose per gram.

| | | Tumour | | | | | | | |
|---|---|---|---|---|---|---|---|---|---|
| | | Delivery | Inorganic | | Active/ | Size | | | |
| Citation | Dose | (% ID/g) | or Organic | Material | Passive | (Small/Big) | Tumor model | Cancer type | Cell line |
| Pathak 2009 | 4.11E+06 | 0.15 | Organic | Polymeric | Passive | Big | Allograft | Breast | Ehrlich ascites |
| | | | | | | | heterotopic | adenocarcinoma | tumor cell |
| Pathak 2009 (2) | 1.29E+07 | 1.17 | Organic | Polymeric | Passive | Big | Allograft | Breast | Ehrlich ascites |
| | | | | | | | heterotopic | adenocarcinoma | tumor cell |
| Wang 2015 | 9.76E+09 | 0.83 | Inorganic | Other | Passive | Smail | Allograft | Breast | 4T1 |
| | | | | | | | heterotopic | | |
| Wu 2013 | 9.85E+09 | 0.38 | Organic | Hydrogel | Passive | Big | Xenograft | Brain | U87MG |
| | | | | | | | heterotopic | | |
| Chakravarty 2015 | 1.80E+10 | 3 | Inorganic | Silica | Passive | Big | Xenograft | Brain | U87MG |
| | | | | | | | heterotopic | | |
| Chakravarty 2015 | 1.80E+10 | 4.5 | Inorganic | Silica | Active | Big | Xenograft | Brain | U87MG |
| (2) | | | | | | | heterotopic | | |
| Behnam Azad | 3.00E+10 | 4.3 | Inorganic | Iron Oxide | Active | Big | Xenograft | Prostate | PSMA |
| 2015 | | | | | | | heterotopic | | |
| Arnida 2011 | 4.70E+10 | 0.3 | Inorganic | Gold | Passive | Big | Xenograft | Ovarian cancer | A2780 |
| | | | | | | | orthotopic | | |
| Chen 2015 | 5.25E+10 | 1.3 | Inorganic | Other | Passive | Big | Xenograft | Breast | MCF-7 |
| | | | | | | | heterotopic | | |
| Chen 2015 (2) | 5.25E+10 | 6 | Inorganic | Other | Active | Big | Xenograft | Breast | MCF-7 |
| | | | | | | | heterotopic | | |
| Chu 2013 (2) | 5.88E+10 | 0.01 | Organic | Polymeric | Passive | Big | Xenograft | Lung | A549 |
| | | | | | | | heterotopic | | |
| Dam 2015 | 7.59E+10 | 2 | Inorganic | Gold | Active | Smail | Xenograft | Breast | MDA-MB-231 |
| | | | | | | | heterotopic | | |
| Kennedy 2011 | l.00E+11 | 0.5 | Inorganic | Gold | Passive | Big | Xenograft | Lymphoblastoid | LCL |
| | | | | | | | heterotopic | | |
| Guo 2013 | 1.51E+11 | 2 | Organic | Polymeric | Passive | Big | Allograft | Breast | 4T1 |
| | | | | | | | heterotopic | | |
| Guo 2013 (2) | 1.51E+11 | 4.3 | Organic | Polymeric | Active | Big | Allograft | Breast | 4T1 |
| | | | | | | | heterotopic | | |
| Dam 2015 (2) | 1.52E+11 | 6 | Inorganic | Goid | Active | Small | Xenograft | Breast | MDA-MB-231 |
| | | | | | | | heterotopic | | |
| Cabral 2011 (4) | 1.53E+11 | 8 | Organic | Polymeric | Passive | Big | Xenograft | Pancreas | BxPC3 |
| | | | | | | | heterotopic | | |
| Cabral 2011 (8) | 1.53E+11 | 4 | Organic | Polymeric | Passive | Big | Xenograft | Pancreas | BxPC3 |
| | | | | | | | heterotopic | | |
| Sykes 2014 (2) | 2.00E+11 | 5 | Inorganic | Gold | Passive | Big | Xenograft | Skin | MDA-MB-435 |
| | | | | | | | orthotopic | | |
| Sykes 2014 (6) | 2.00E+11 | 9 | Inorganic | Gold | Active | Big | Xenograft | Skin | MDA-MB-435 |
| | | | | | | | orthotopic | | |
| Chu 2013 | 3.71E+11 | 0.19 | Organic | Polymeric | Passive | Big | Xenograft | Lung | A549 |
| | | | | | | | heterotopic | | |
| Sykes 2014 (3) | 6.00E+11 | 15 | Inorganic | Gold | Passive | Big | Xenograft | Skin | MDA-MB-435 |
| | | | | | | | orthotopic | | |
| Sykes 2014 (7) | 6..00E+11 | 22 | inorganic | Gold | Active | Big | Xenograft | Skin | MDA-MB-435 |
| | | | | | | | orthotopic | | |
| Cabral 2011 (3} | 7.17E+11 | 10 | Organic | Polymeric | Passive | Big | Xenograft | Colon | C26 |
| | | | | | | | heterotopic | | |
| Cabral 2011 (7) | 7.17E+11 | 4 | Organic | Polymeric | Passive | Big | Xenograft | Pancreas | BxPC3 |
| | | | | | | | heterotopic | | |
| Shah 2012 | 8.05E+11 | 3 | Inorganic | Goid | Passive | Big | Xenograft | Prostate | LNCaP |
| | | | | | | | heterotopic | | |
| Amida 2011 (2) | 8.80E+11 | 1.8 | Inorganic | Gold | Passive | | Xenograft | Ovarian cancer | A2780 |
| | | | | | | | orthotopic | | |
| Gormley 2011 | 8.90E+11 | 7.80E+00 | Inorganic | Gold | Passive | | Xenograft | Pancreas | Panc-1 |
| | | | | | | | heterotopic | | |
| Gormley 2011 (2) | 3.90E+11 | 1.56E+00 | Inorganic | Gold | Active | | Xenograft | Pancreas | Panc-1 |
| | | | | | | | heterotopic | | |
| Wu 2015 | 9.92E+11 | 9 | Organic | Hydrogel | Passive | Big | Allograft | Hepatoma | H22 |
| | | | | | | | heterotopic | | |
| Chen 2012 | 1.15E+12 | 4 | Inorganic | Silica | Passive | Big | Allograft | Breast | 4T1 |
| | | | | | | | heterotopic | | |
| Chen 2008 | 1.2E+12 | 0.7 | Inorganic | Other | Passive | Small | Allograft | Colon | C26 |
| | | | | | | | heterotopic | adenocarinoma | |
| Chen 2008 (2) | 1.2E+12 | 4 | Inorganic | Other | Active | Small | Allograft | Colon | C26 |
| | | | | | | | heterotopic | adenocarinoma | |
| Cai 2007 | 1.20E+12 | 0.7 | Inorganic | Other | Passive | Small | Xenograft | Brain | U87MG |
| | | | | | | | heterotopic | | |
| Cai 2007 (2) | 1.20E+12 | 4.3 | Inorganic | Other | Active | Small | Xenograft | Brain | U87MG |
| | | | | | | | heterotopic | | |
| Wang 2014 | 1.45E+12 | 2.5 | Organic | Hydrogel | Passive | Big | Allograft | Hepatoma | H22 |
| | | | | | | | heterotopic | | |
| Wang 2014 (2) | 1.45E+12 | 2.5 | Organic | Hydrogel | Active | Big | Allograft | Hepatoma | H22 |
| | | | | | | | heterotopic | | |
| Goodrich 2010 | 1.60E+12 | 1.92 | Inorganic | Gold | Passive | Small | Allograft | Colon | C26 |
| | | | | | | | heterotopic | adenocarinoma | |
| Cabral (6) | 1.68E+12 | 7 | Organic | Polymeric | Passive | Big | Xenograft | Colon | C26 |
| | | | | | | | heterotopic | adenocarinoma | |
| Cabral 2011 (2) | 1.68E+12 | 9 | Organic | Polymeric | Passive | Big | Xenograft | Colon | C26 |
| | | | | | | | heterotopic | adenocarinoma | |
| Sykes 2014 (4) | 2.00E+12 | 18 | Inorganic | Gold | Passive | Big | Xenograft | Skin | MDA-MB-435 |
| | | | | | | | orthotopic | | |
| Sykes 2014 (8) | 2.00E+12 | 27 | Inorganic | Gold | Active | Big | Xenograft | Skin | MDA-MB-435 |
| | | | | | | | orthotopic | | |
| Liu 2014 | 2.40E+12 | 1 | Inorganic | Gold | Passive | Small | Xenograft | Cervical | KB |
| | | | | | | | heterotopic | | |
| Cheng | 2.40E+12 | 2.5 | Inorganic | Gold | Passive | Small | Xenograft | Brain | U87MG |
| | | | | | | | heterotopic | | |
| Cheng (2) | 2.40E+12 | 8 | Inorganic | Gold | Active | Small | Xenograft | Brain | U87MG |
| | | | | | | | heterotopic | | |
| Wong 2013 | 3.00E+12 | 6 | Organic | Liposomes | Passive | Big | Allograft | Breast | MET1 |
| | | | | | | | orthotopic | | |
| Liu 2007 | 3.61E+12 | 3 | Organic | Other | Passive | | Xenograft | Brain | U87MG |
| | | | | | | | heterotopic | | |
| Liu 2007 (2) | 3.61E+12 | 3 | Organic | Other | Passive | | Xenograft | Brain | U87MG |
| | | | | | | | heterotopic | | |
| Liu 2007 (3) | 3.61E+12 | 13 | Organic | Other | Active | | Xenograft | Brain | U87MG |
| | | | | | | | heterotopic | | |
| Hu 2015 | 4E+12 | 6.49 | Organic | Polymeric | Passive | Small | Xenograft | Brain | U87MG |
| | | | | | | | heterotopic | | |
| Negi 2014 | 5.20E+12 | 3 | Organic | Liposomes | Passive | Big | Allograft | Breast | Ehrlich ascites |
| | | | | | | | heterotopic | adenocarcinoma | tumor cell |
| Perez-Medina | 5.43E+12 | 13.7 | Organic | Liposomes | Passive | Big | Allograft | Breast | 4T1 |
| 2014 | | | | | | | heterotopic | | |
| Kirpotin 2006 | 6.76E+12 | 8 | Organic | Liposomes | Passive | Big | Xenograft | Breast | BT474 |
| | | | | | | | heterotopic | | |
| Kirpotin 2006 (2) | 6.76E+12 | 8 | Organic | Liposomes | Active | Big | Xenograft | Breast | BT474 |
| | | | | | | | heterotopic | | |
| Zhong 2015 | 7.12E+12 | 17 | Inorganic | Gold | Passive | Small | Xenograft | Cervical | HeLA |
| | | | | | | | heterotopic | | |
| Chang 2010 | 7.17E+12 | 6.1 | Organic | Liposomes | Passive | Big | Allograft | Colon | C26 |
| | | | | | | | heterotopic | adenocarinoma | |
| Okuda 2006 | 7.17E+12 | 14.5 | Organic | Hydrogel | Passive | Small | Allograft | Colon | C26 |
| | | | | | | | heterotopic | adenocarinoma | |
| Shi 2015 | 9.61E+12 | 8 | Organic | Polymeric | Passive | Big | Xenograft | Skin | A431 |
| | | | | | | | orthotopic | | |
| Sykes 2014 | 1.00E+13 | 20 | Inorganic | Gold | Passive | Small | Xenograft | Skin | MDA-MB-435 |
| | | | | | | | orthotopic | | |
| Sykes 2014 (5) | 1.00E+13 | 30 | Inorganic | Gold | Active | Small | Xenograft | Skin | MDA-MB-435 |
| | | | | | | | orthotopic | | |
| Lee 2011 | 1.20E+13 | 7.91 | Organic | Liposomes | Passive | Big | Xenograft | Brain | U87MG |
| | | | | | | | heterotopic | | |
| Song 2014 | 1.38E+13 | 8 | Organic | Liposomes | Passive | Big | Allograft | Breast | T11 gem |
| | | | | | | | orthotopic | | |
| Cabral 2011 (1) | 1.39E+13 | 10 | Organic | Polymeric | Passive | Small | Xenograft | Colon | C26 |
| | | | | | | | heterotopic | adenocarinoma | |
| Cabral 2011 (5) | 1.39E+13 | 11 | Organic | Polymeric | Passive | Small | Xenograft | Pancreas | BxPC3 |
| | | | | | | | heterotopic | | |
| Khalid 2006 | 6.16E+13 | 10.5 | Organic | Liposomes | Passive | Big | Allograft | Colon | C26 |
| | | | | | | | heterotopic | adenocarinoma | |
| DeNardo 2007 | 1.04E+14 | 12.5 | Inorganic | Iron Oxide | Active | Small | Xenograft | Breast | HBT3477 |
| | | | | | | | heterotopic | | |
| Zolata 2014 | 3E+16 | 10 | Inorganic | Gold/iron | Active | Small | Xenograft | Breast | SKBR3 |
| | | | | | | | heterotopic | | |

TABLE 2
We were unable to calculate doses of these studies and so we excluded
them from the analysis. The reasons for exclusion are given.

| Study | |
|---|---|
| (Author, year) | Reasons for exclusion |
| Zhang 2015 | Ultrasmall; exclude |
| Meyers 2015 | Pc4 dose given; gold dose unclear |
| Hu 2014 | Dosing given in radioactivity, not particles |
| Razzak 2013 | Can't find reference |
| Zhang 2015 | Ultrasmall; exclude |
| Black 2014 | Only reported radioactive dose; unclear how gold dose |
| | determined |
| Liu 2013 | Ultrasmall; exclude |
| Karmani 2013 | Only reported radioactive dose; unclear how gold dose |
| | determined |
| Wang 2012 | Only reported radioactive dose; unclear how gold dose |
| | determined |
| Perrault 2009 | Can't figure out the delivery from the log graphs |
| Yang 2013 | Dose unclear; it's a mixed element nanoparticle |
| Chauhan 2013 | Excluded b/c values don't make sense (see blood in |
| | FIG. 9a) |
| Chauhan 2013 | Unclear conjugation/radiolabelling amounts |
| Yang 2011 | Unclear conjugation/labelling amounts |
| Quan 2011 | Signal appears to be noise |
| Goel 2014 | Unclear conjugation/radiolabelling amounts |
| Chen 2014 | Unclear conjugation/radiolabelling amounts |
| Chen 2014 | Unclear conjugation/radiolabelling amounts |
| Benezra 2011 | Total accumulation in all organs is <10% at 24 h; |
| | excluded. |
| Chen 2013 | Unclear conjugation/radiolabelling amounts |
| Hu 2013 | Total accumulation in all organs is <10% at 24 h; |
| | excluded. |
| Yu 2015 | Unconventional shape and hard to calculate dose |
| Zhang 2013 | ip injection; exclude |
| Mi 2014 | Unclear how many Gd per CaP nanoparticle; exclude |
| Huang 2015 | Dose unclear |
| Hong 2015 | 0.97 GBq/mg; unclear what the size is; exclude |
| Al-Jamal 2009 | Unclear how many QD per liposome |
| Kai 2015 | Only relative AUC is given and not sure what the % |
| | ID is; exclude |
| Chen 2015 | Recovery <10%; exclude |
| Oliveira 2014 | Bunch of AUCs and unclear what % ID/g is; exclude |
| Polyak 2013 | Rat; unclear conjugation/radiolabelling amounts; exclude |
| Ding 2013 | Recovery <10%; exclude |
| Chu 2013 | Only AUC given; Unclear the % ID/g; exclude |
| Ma 2012 | Recovery <10%; exclude |
| Guo 2013 | 4.6 wt % dox; recovery <10%; exclude |
| Sumitani 2011 | Unclear dose; exclude |
| Bae 2007 | Unclear dose (mmol wt % ???); exclude |
| Cabral 2004 | Dose unclear |
| Bibby 2005 | Dox is 3.3 wt %; thus 7.6 mg of polymer injected per |
| | mouse; Size/density unclear - exclude |
| Bae 2005 | Loading effciency and density unclear; exclude |
| van Vlerken | Recovery <10% and <24 h study; exclude |
| 2008 | |
| Sasatsu 2008 | Density unclear; exclude |
| Rossin 2005 | Density/radiolabelling unclear; exclude |
| Mondal 2010 | 94% radiolabelling effociency; dose of particle unclear |
| He 2010 | Density unclear; exclude |
| Cabral 2007 | Loading efficiency and density unclear; exclude |
| Shi 2013 | Unclear conjugation/radiolabelling amounts |
| Hong 2012 | Unclear conjugation/radiolabelling amounts |
| Xu 2015 | Recovery <10% + unclear size dimensions; exclude |
| Shi 2014 | Unclear conjugation/radiolabelling amounts |
| Hong 2012 | Unclear conjugation/radiolabelling amounts |
| Lin 2014 | Unclear conjugation/radiolabelling amounts |
| Hirsjarvi 2013 | Unclear what ingredients they're using to calculate dose |
| Lin 2011 | Unclear how to convert phospholipids to liposomes |
| Miyajima 2006 | Unclear what concentration was |
| Paolino 2010 | DPPC:Chol:PEG at 6:3:1; recovery <10% = exclude |
| Zamboni 2007 | Unclear loading amount - exclude |
| Chen 2008 | IP administration; exclude |
| Yuan 2006 | Recovery <10%; exclude |
| Chen 2010 | Unclear radiolabelling amount; exclude |
| Chang 2007 | Unclear radiolabelling amount; exclude |
| Han 2015 | Unclear liposome ingredients and loading ratios |
| Yang 2015 | 16% wt loading; Recovery <10% exclude |
| Ganesh 2013 | Recovery <10% exclude |
| Xu 2013 | Unclear accumulation at 24 h (log scales); exclude |
| Cheng 2012 | Recovery <10% exclude |
| Kommareddy | Unclear radiolabelling |
| 2007 | |
| Qian 2014 | Recovery <10% exclude |
| Sadekar 2011 | Recovery <10% exclude |
| Kukowska- | Recovery <10% exclude |
| Latallo 2005 | |
| Zhang 2015 | Rats; exclude |
| Chen 2015 | Unclear gold:particle ratio; exclude |
| Balogh 2007 | Unclear dose; exclude |
| Sadekar 2012 | Unclear dose; exclude |
| Tian 2015 | Unclear size/doses; exclude |
| Kim 2015 | Recovery <10%; exclude |
| Harivardhan | Recovery <10%; exclude |
| 2005 | |
| Lee 2013 | Unclear density of upconverting particle; excluded |

TABLE 3
Output tables from SPSS multiple regression
analysis of Wilhelm et al, Nat Rev Mat, 2016.
3a. Model Summary

| | | | | | Change Statistics | | | | |
|---|---|---|---|---|---|---|---|---|---|
| | | | Adjusted | Std. Error | R | | | | |
| | | R | R | of the | Square | F | | | Sig. F |
| Model | R | Square | Square | Estimate | Change | Change | df1 | df2 | Change |
| 1 | 0.711 | 0.506 | 0.434 | 4.83227 | 0.506 | 7.036 | 8 | 55 | 0.000 |

3b. Coefficients of the model.
The model is of the general form
delivery = βᵢxᵢ + constant
where xᵢ are the variables listed below and βᵢ are their corresponding coefficients:

| | Unstandardized | | | | | 95.0% Confidence | |
|---|---|---|---|---|---|---|---|
| | Coefficients | | Standardized | | | Interval for B | |
| | | Std. | Coefficients | | | Lower | Upper |
| Variable | B | Error | Beta | t | Sig. | Bound | Bound |
| (Constant) | −37.554 | 9.337 | | −4.022 | 0.00000 | −56.265 | −18.843 |
| Dose (log) | 3.395 | 0.734 | 0.48 | 4.622 | 0.00002 | 1.923 | 4.867 |
| Cancer | 0.565 | 0.199 | 0.368 | 2.844 | 0.00600 | 0.167 | 0.963 |
| Type | | | | | | | |
| Active vs | −3.918 | 1.454 | −0.276 | −2.696 | 0.00900 | −6.831 | −1.005 |
| Passive | | | | | | | |
| Size | 1.504 | 1.073 | 0.142 | 1.402 | 0.16700 | −0.646 | 3.653 |
| Tumor | 0.896 | 0.832 | 0.143 | 1.077 | 0.28600 | −0.771 | 2.564 |
| Model | | | | | | | |
| Cell Line | 0.083 | 0.107 | 0.098 | 0.779 | 0.44000 | −0.131 | 0.298 |
| Inorganic | 0.705 | 1.735 | 0.055 | 0.406 | 0.68600 | −2.773 | 4.183 |
| vs Organic | | | | | | | |
| Material | −0.098 | 0.316 | −0.039 | −0.311 | 0.75700 | −0.733 | 0.536 |

### REFERENCES

1 Wilhelm, S. et al. Analysis of nanoparticle delivery to tumours. Nat. Rev. Mater. 1, 1-12 (2016).
2. Zhang, Y.-N., Poon, W., Tavares, A. J., McGilvray, I. D. & Chan, W. C. W. Nanoparticle—liver interactions: Cellular uptake and hepatobiliary elimination. J. Control. Release 240, 332-348 (2016).
3. Tsoi, K. M. et al. Mechanism of hard-nanomaterial clearance by the liver. Nat. Mater. 15, 1212-1221 (2016).
4. Park, J.-K. et al. Cellular distribution of injected PLGA-nanoparticles in the liver. Nanomedicine Nanotechnology, Biol. Med. 12, 1365-1374 (2016).
5. Rodriguez, P. L. et al. Minimal ‘Self’ peptides that inhibit phagocytic clearance and enhance delivery of nanoparticles. Science 339, 971-5 (2013).
6. Patel, P. C. et al. Scavenger receptors mediate cellular uptake of polyvalent oligonucleotide-functionalized gold nanoparticles. Bioconjug. Chem. 21, 2250-2256 (2010).
7. Ohta, S., Glancy, D. & Chan, W. C. W. DNA-controlled dynamic colloidal nanoparticle systems for mediating cellular interaction. Science 351, 841-845 (2016).
8. Liu, D., Mori, A. & Huang, L. Role of liposome size and RES blockade in controlling biodistribution and tumor uptake of GM1-containing liposomes. BBA-Biomembr. 1104, 95-101 (1992).
9. Rahman, Y. E., Cerny, E. A., Patel, K. R., Lau, E. H. & Wright, B. J. Differential uptake of liposomes varying in size and lipid composition by parenchymal and kupffer cells of mouse liver. Life Sci. 31, 2061-2071 (1982).
10. Perrault, S. D., Walkey, C., Jennings, T., Fischer, H. C. & Chan, W. C. W. Mediating tumor targeting efficiency of nanoparticles through design. Nano Lett. 9, 1909-1915 (2009).
11. Arnida, Janat-Amsbury, M. M., Ray, A., Peterson, C. M. & Ghandehari, H. Geometry and surface characteristics of gold nanoparticles influence their biodistribution and uptake by macrophages. Eur. J. Pharm. Biopharm. 77, 417-423 (2011).
12. Huang, X. et al. The shape effect of mesoporous silica nanoparticles on biodistribution, clearance, and biocompatibility in vivo. ACS Nano 5, 5390-5399 (2011).
13. Juliano, R. L. & Stamp, D. The effect of particle size and charge on the clearance rates of liposomes and liposome encapsulated drugs. Biochem. Biophys. Res. Commun. 63, 651-658 (1975).
14. Niidome, T. et al. PEG-modified gold nanorods with a stealth character for in vivo applications. J. Control. Release 114, 343-347 (2006).
15. Albanese, A., Tang, P. S. & Chan, W. C. W. The Effect of Nanoparticle Size, Shape, and Surface Chemistry on Biological Systems. Annu. Rev. Biomed. Eng. 14, 1-16 (2012).
16. Wolfram, J. et al. A chloroquine-induced macrophage-preconditioning strategy for improved nanodelivery. Sci. Rep. 7, 1-13 (2017).
17. Tavares, A. J. et al. Effect of removing Kupffer cells on nanoparticle tumor delivery. Proc. Natl. Acad. Sci. U. S. A. 114, E10871-E10880 (2017).
18. lio, M. & Wagner, H. N. Studies of the Reticuloendothelial System (RES). I

Measurement of the Phagocytic Capacity of the RES in Man and Dog. J. Clin. Invest. 42, 417-426 (1963).
19. Sun, X. et al. Molecular imaging of tumor-infiltrating macrophages in a preclinical mouse model of breast cancer. Theranostics 5, 597-608 (2015).
20. Liu, T., Choi, H., Zhou, R. & Chen, I. W. RES blockade: A strategy for boosting efficiency of nanoparticle drug. Nano Today 10, 11-21 (2015).
21. Allen, T. M. & Hansen, C. Pharmacokinetics of stealth versus conventional liposomes: effect of dose. BBA-Biomembr. 1068, 133-141 (1991).
22. Panagi, Z. et al. Effect of dose on the biodistribution and pharmacokinetics of PLGA and PLGA-mPEG nanoparticles. Int. J. Pharm. 221, 143-152 (2001).
23. Gabizon, A., Tzemach, D., Mak, L., Bronstein, M. & Horowitz, A. T. Dose dependency of pharmacokinetics and therapeutic efficacy of pegylated liposomal doxorubicin (DOXIL) in murine models. J. Drug Target. 10, 539-548 (2002).
24. Laverman, P. et al. Preclinical and clinical evidence for disappearance of long-circulating characteristics of polyethylene glycol liposomes at low lipid dose. J. Pharmacol. Exp. Ther. 293, 996-1001 (2000).
25. Utkhede, D. R. & Tilcock, C. P. Effect of Lipid Dose on the Redistribution and Blood Pool Clearance Kinetics of Peg-Modified Technetium-Labeled Lipid Vesicles. 2104, (2008).
26. Faria, M. et al. Minimum information reporting in bio-nano experimental literature. Nat. Nanotechnol. 13, 777-785 (2018).
27. Sun, X. et al. Improved tumor uptake by optimizing liposome based res blockade strategy. Theranostics 7, 319-328 (2017).
28. Proffitt, R. T. et al. Liposoinal Blockade of the Reticuloendothelial System : Improved Tumor Imaging with Small Unilamellar Vesicles. Science (80-.). 220, 502-505 (1983).
29. Kai, M. P. et al. Tumor presence induces global immune changes and enhances nanoparticle clearance. ACS Nano 10, 861-870 (2016).
30. Bouwens, L., Baekeland, M., de Zanger, R. & Wisse, E. Quantitation, tissue distribution and proliferation kinetics of kupffer cells in normal rat liver. Hepatology 6, 718-722 (1986).
31. Lee, S.-H., Starkey, P. M. & Gordon, S. Quantitative Analysis of Total Macrophage Content in Adult Mouse Tissues: Immunochemical Studies with Monoclonal Antibody F4/80. J. Exp. Med. 161, 475-489 (1985).
32. Poon, W. et al. Elimination Pathways of Nanoparticles. ACS Nano 13, 5785-5798 (2019).
33. Ishida, T. & Kiwada, H. Accelerated blood clearance (ABC) phenomenon upon repeated injection of PEGylated liposomes. Int. J. Pharm. 354, 56-62 (2008).
34. Ishida, T., Atobe, K., Wang, X. Y. & Kiwada, H. Accelerated blood clearance of PEGylated liposomes upon repeated injections: Effect of doxorubicin-encapsulation and high-dose first injection. J. Control. Release 115, 251-258 (2006).
35. Ishida, T. et al. Injection of PEGylated liposomes in rats elicits PEG-specific IgM, which is responsible for rapid elimination of a second dose of PEGylated liposomes. J. Control. Release 112, 15-25 (2006).
36. Ishida, T. et al. Accelerated blood clearance of PEGylated liposomes following preceding liposome injection: Effects of lipid dose and PEG surface-density and chain length of the first-dose liposomes. J. Control. Release 105, 305-317 (2005).
37. Sercombe, L. et al. Advances and challenges of liposome assisted drug delivery. Front. Pharmacol. 6, 1-13 (2015).
38. Sasmono, R. T. et al. A macrophage colony-stimulating factor receptor-green fluorescent protein transgene is expressed throughout the mononuclear phagocyte system of the mouse. Blood 101, 1155-1163 (2003).
39. Kolb-Bachofen, V., Schlepper-Schäfer, J. & Kolb, H. Receptor-mediated particle uptake by liver macrophages. Exp. Cell Res. 148, 173-182 (1983).
40. Commisso, C. et al. Macropinocytosis of protein is an amino acid supply route in Ras-transformed cells. Nature 497, 633-637 (2013).
41. Canton, J. et al. Calcium-sensing receptors signal constitutive macropinocytosis and facilitate the uptake of NOD2 ligands in macrophages. Nat. Commun. 7, (2016).
42. Walkey, C. D., Olsen, J. B., Guo, H., Emili, a & Chan, W. C. Nanoparticle size and surface chemistry determine serum protein adsorption and macrophage uptake. J Am Chem Soc 134, 2139-2147 (2012).
43. Gordon, S. Phagocytosis: An Immunobiologic Process. Immunity 44, 463-475 (2016).
44. van Furth, R. et al. The mononuclear phagocyte system: a new classification of macrophages, monocytes, and their precursor cells. Bull. World Health Organ. 46, 845-852 (1972).
45. Minchinton, A. I. & Tannock, I. F. Drug penetration in solid tumours. Nat. Rev. Cancer 6, 583-592 (2006).
46. Sykes, E. a, Chen, J., Zheng, G. & Chan, W. C. W. Investigating the Impact of Nanoparticle Size on Investigating the Impact of Nanoparticle Size on Active and Passive Tumor Targeting Efficiency. ACS Nano 8, 5696-5706 (2014).
47. Kingston, B. R., Syed, A. M., Ngai, J., Sindhwani, S. & Chan, W. C. W. Assessing micrometastases as a target for nanoparticles using 3D microscopy and machine learning. Proc. Natl. Acad. Sci. U. S. A. 116, 14937-14946 (2019).
48. Syed, A. M. et al. Three-Dimensional Imaging of Transparent Tissues via Metal Nanoparticle Labeling. J. Am. Chem. Soc. 139, 9961-9971 (2017).
49. Seymour, L. W. et al. Tumour tropism and anti-cancer efficacy of polymer-based doxorubicin prodrugs in the treatment of subcutaneous murine B16F10 melanoma. Br. J. Cancer 70, 636-641 (1994).
50. Ohara, Y. et al. Effective delivery of chemotherapeutic nanoparticles by depleting host Kupffer cells. Int. J. Cancer 131, 2402-2410 (2012).
51. Charrois, G. J. R. & Allen, T. M. Multiple injections of pegylated liposomal doxorubicin: Pharmacokinetics and therapeutic activity. J. Pharmacol. Exp. Ther. 306, 1058-1067 (2003).
52. Griffin, J. I. et al. Revealing Dynamics of Accumulation of Systemically Injected Liposomes in the Skin by Intravital Microscopy. ACS Nano 11, 11584-11593 (2017).
53. Simberg, D. et al. Biomimetic amplification of nanoparticle homing to tumors. 104, (2007).
54. Souhami, R. L., Patel, H. M. & Ryman, B. E. The effect of reticuloendothelial blockade on the blood clearance and tissue distribution of liposomes. BBA-Gen. Subj. 674, 354-371 (1981).
55. Fernández-Urrusuno, R. et al. Effect of polymeric nanoparticle administration on the clearance activity of the mononuclear phagocyte system in mice. J. Biomed. Mater. Res. 31, 401-408 (1996).
56. Wagner, H. N. & lio, M. Studies of the Reticuloendothelial System (RES). III Blockade of the RES in Man. J. Clin. Invest. 43, 1525-1532 (1964).
57. Mcneil, S. E. Evaluation of nanomedicines : stick to the basics. Nat. Rev. Mater. 1, (2016).
58. Wilhelm, S., Tavares, A. J. & Chan, W. C. W. Reply to ‘“Evaluation of nanomedicines: stick to the basics”’. Nat. Rev. Mater. 1, 1-2 (2016).
59. Hamaguchi, T. et al. NK105, a paclitaxel-incorporating micellar nanoparticle formulation, can extend in vivo antitumour activity and reduce the neurotoxicity of paclitaxel. Br. J. Cancer 92, 1240-1246 (2005).
60. Hamaguchi, T. et al. A phase I and pharmacokinetic study of NK105, a paclitaxel-incorporating micellar nanoparticle formulation. Br. J. Cancer 97, 170-176 (2007).
61. Fujiwara, Y. et al. A multi-national, randomised, open-label, parallel, phase III non-inferiority study comparing NK105 and paclitaxel in metastatic or recurrent breast cancer patients. Br. J. Cancer 120, 475-480 (2019).
62. Autio, K. A. et al. Safety and Efficacy of BIND-014, a Docetaxel Nanoparticle Targeting Prostate-Specific Membrane Antigen for Patients with Metastatic Castration-Resistant Prostate Cancer: A Phase 2 Clinical Trial. JAMA Oncol. 4, 1344-1351 (2018).
63. van der Meel, R. et al. Smart cancer nanomedicine. Nat. Nanotechnol. 14, 1007-1017 (2019).
64. Rangarajan, S. et al. AAV5-Factor VIII Gene Transfer in Severe Hemophilia A. N. Engl. J. Med. 377, 2519-2530 (2017).
65. Sindhwani, S. et al. The entry of nanoparticles into solid tumours. Nat. Mater. (2020) doi:10.1038/s41563-019-0566-2.
66. Szebeni, J., Simberg, D., Gonzalez-Fernandez, A., Barenholz, Y. & Dobrovolskaia, M. A. Roadmap and strategy for overcoming infusion reactions to nanomedicines. Nat. Nanotechnol. 13, 1100-1108 (2018).
67. La-beck, N. M. & Gabizon, A. A. Nanoparticle interactions with the immune system : clinical implications for Liposome-Based cancer chemotherapy. 8, 6-11 (2017).

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

What is claimed is:

1. A composition comprising a mixture of: (a) first nanoparticle carrier vehicles loaded with an active ingredient for treating a disorder (therapeutic nanoparticles), and (b) second nanoparticle carrier vehicles without the active ingredient (decoy nanoparticles), the nanoparticle carrier vehicles being one of gold nanoparticles, liposomes, silica nanoparticles, micelles, nanogel or polymeric nanoparticles.

2. The composition of claim 1, wherein the composition is provided as a combined single dose for humans of therapeutic nanoparticles and decoy nanoparticles of at least one and one-half (1.5) quadrillion (10¹⁵) nanoparticles.

3. The composition of claim 1, wherein other than the presence of the active ingredient in the therapeutic nanoparticles, a chemical composition of the therapeutic nanoparticles is substantially identical to a chemical composition of the decoy nanoparticles.

4. The composition of claim 1, wherein the therapeutic nanoparticle is loaded with a single active ingredient for treating or preventing a disorder.

5. The composition according to claim 1, wherein the decoy nanoparticle is devoid of any biologically active ingredient.

6. The composition according to claim 1, wherein the composition comprises a higher ratio of decoy:therapeutic nanoparticles to stoichiometrically force the decoy nanoparticles to bind to liver cells responsible for nanoparticle clearance thereby allowing the therapeutic nanoparticles for continued circulation.

7. The composition according to claim 1, wherein both the therapeutic and decoy nanoparticles include a polyethylene glycol coating.

8. A medicament for treating or preventing a disease, disorder or condition, wherein the medicament comprises a composition according to claim 1, and wherein the active ingredient in the therapeutic nanoparticles is effective for treating or preventing the disease, disorder or condition.

9. The medicament of claim 8, wherein the disease, disorder or condition is cancer.

10. A kit comprising: (a) a composition comprising first nanoparticle carrier vehicles loaded with an active ingredient for treating or preventing a disease, disorder or condition (therapeutic nanoparticles), (b) a composition comprising second nanoparticle carrier vehicles without the active ingredient (decoy nanoparticles), and (c) instruction for administering composition (a) and composition (b), the nanoparticle carrier vehicles being one of silica nanoparticles, liposomes, gold nanoparticles, micelles, nanogel or polymeric nanoparticles.

11. The kit of claim 10, wherein composition (a) and composition (b) are provided as a mixture.

12. The kit of claim 10, wherein the instructions include instructions for simultaneously co-administering compositions (a) and (b).

13. The kit according to claim 10, wherein the chemical composition of first nanoparticle carrier vehicles in (a) is substantially similar or substantially identical to the chemical composition of the second nanoparticle carrier vehicles in (b).

14. The kit according to claim 10, wherein the instructions comprises a single combined dose for humans of therapeutic nanoparticles and decoy nanoparticles of at least one and one-half (1.5) quadrillion (10¹⁵) nanoparticles.

15. The kit according to claim 10, wherein the instruction includes administering a higher ratio of decoy:therapeutic nanoparticles to stoichiometrically force the decoy nanoparticles to bind to liver cells responsible for nanoparticle clearance thereby allowing the therapeutic nanoparticles for continued circulation

16. The kit according to claim 10, wherein the first and second lipid based carrier vehicles include PEGylation.

17. A use of the composition according to claim 1, to treat or prevent a disease, disorder or condition.

18. The use of claim 17, wherein the disease, disorder or condition is cancer.

19. A method of increasing dose efficacy or delivery efficiency for a therapeutic treatment of a disease, disorder or condition in a subject, the method comprising simultaneously co-administering to the subject a medicament of claim 8.

20. The method of claim 19, wherein the disease, disorder or condition is cancer.

21. A method of treating a disease, disorder or condition, the method comprising simultaneously co-administering to a subject in need a medicament of claim 8.

22. The method of claim 21, wherein the disease, disorder or condition is cancer.

23. A method of treating or preventing a disease, disorder or condition, the method comprising administering to a human subject in need a dose of a composition having nanoparticles containing an active ingredient effective to treat or prevent the disease, disorder or condition, wherein the dose is in terms of number of nanoparticles, and wherein the nanoparticles is one of gold nanoparticles, liposomes, silica nanoparticles, micelles, nanogel or polymeric nanoparticles.

24. The method of claim 23, wherein the number of nanoparticles in the dose is of at least one and one-half (1.5) quadrillion (10¹⁵) nanoparticles.

25. The method of claim 23, where the dose is provided as a single dose.
